(19) United States
(12) Patent Application Publication
(10) Pub. No.: US 2007/0184547 A1
(43) Pub. Date: Aug. 9, 2007

(54) Polynucleotide sample preparation device

(76) Inventors: Kalyan Handique, Ypsilanti, MI (US); Jeff Williams, Chelsea, MI (US); Sundaresh N. Brahmasandra, Ann Arbor, MI (US); Nikhil Phadke, Ann Arbor, MI (US); Betty Wu, Canton, MI (US)

Correspondence Address: FISH & RICHARDSON P.C., PO BOX 1022, MINNEAPOLIS, MN, 55440-1022, US

(21) Appl. No.: 11/580,267

(22) Filed: Oct. 11, 2006

Related U.S. Application Data
(60) Provisional application No. 60/726,066, filed on Oct. 11, 2005.

(51) Int. Cl. C12M 1/34; C12P 19/34

(52) U.S. Cl. 435/288.5; 435/91.2; 435/287.2; 435/303.1

(57) ABSTRACT

Methods and systems for preparing polynucleotide samples are disclosed. The invention includes a microfluidic system for converting a sample containing one or more polynucleotides into a form suitable for analyzing the polynucleotides, comprising: a cartridge receiving element, an insertable and removable cartridge, a heating element configured to heat one or more regions of the cartridge, and control circuitry, wherein the insertable cartridge comprises: a microfluidic component that is configured to accept the sample and one or more reagents, and to react the sample and the reagents, in order to produce a prepared sample suitable for analyzing the one or more polynucleotides. The invention further comprises a multi-sample cartridge for converting a number of samples, each containing one or more polynucleotides, into respective forms suitable for analyzing the polynucleotides, comprising: at least a first microfluidic component and a second microfluidic component.

### CROSS-REFERENCE TO RELATED APPLICATIONS

The present application claims the benefit of priority of U.S. provisional application Ser. No. 60/726,066, filed Oct. 11, 2005, the specification of which is incorporated herein by reference in its entirety.

### TECHNICAL FIELD

This technology described herein relates to methods and devices for preparing polynucleotide-containing samples, and more particularly to methods and devices that utilize microfluidic components for preparing samples for subsequent analysis of polynucleotides contained therein.

### BACKGROUND

Many laboratory techniques involve detection, quantitative analysis, or amplification of polynucleotides. For example, the polymerase chain reaction (PCR) is a well-established routine laboratory practice for amplifying DNA in DNA-containing samples. Nevertheless, even routine practices would benefit from levels of automation that would increase throughput, improve consistency of analyses, and be simple to use, as well as save processing and analysis time for individual samples.

One aspect in which the overall time of an analysis, such as PCR, can be significantly shortened, without a detrimental impact on reliability, is the initial processing of the nucleotide-containing sample. Since analytical techniques such as PCR have already been subject to certain levels of automation within the industry, there exists a need to develop efficient means of sample preparation that provides DNA extracts from raw clinical samples in a form that can be immediately input to existing machines.

For analytic methods such as PCR to be effective, individual DNA molecules must be liberated from their host cell nuclei. Thus, in cell-containing samples, cell walls, and nuclear membranes must both be ruptured to permit DNA molecules to enter the surrounding milieu. Overall, several steps are typically required to extract useable DNA from a cell-containing sample. Development of a simple device that can carry out such steps routinely and efficiently would be of considerable benefit to, for example, those who carry out existing PCR protocols, not least because existing attempts at automation have involved complex and expensive technologies, such as robotics.

Microfluidics has proven to be a practical technology for carrying out both sample preparation for diagnostic analysis, and analysis of micro-liter scale samples by methods such as PCR. See, for example, PCT application no., PCT/US2005/015345, and U.S. provisional application Nos. 60/567,174, and 60/645,784, all of which are incorporated herein by reference in their entirety. However, to date, a tool that has not been developed is a microfluidic component that can deliver nucleotide samples in a form that can be conveniently analyzed by existing laboratory equipment, including the thermal cyclers used in PCR.

Microfluidic devices with various components are described in U.S. provisional application No. 60/553,553 filed Mar. 17, 2004 by Parunak et al., which is incorporated herein by reference.

### SUMMARY

Systems as described herein include a microfluidic system for converting a sample containing one or more polynucleotides into a form suitable for analyzing the one or more polynucleotides, the system comprising: a cartridge receiving element in communication with an insertable and removable cartridge; a heating element in communication with the cartridge receiving element, configured to heat one or more regions of the cartridge; and control circuitry in communication with the heating element; wherein the insertable cartridge comprises: at least one microfluidic component that, in conjunction with the heating element and the control circuitry, is configured to accept the sample and one or more reagents, and to react the sample and the reagents, in order to produce a prepared sample suitable for analysis of the one or more polynucleotides.

In other embodiments, the insertable cartridge further comprises: a sample inlet for receiving the sample; a reagent inlet for accepting one or more reagents; and an outlet for directing prepared sample into a PCR tube. In still other embodiments, the microfluidic component comprises: one or more channels configured to transmit volumes of fluid in the range 0.1-50 μl, wherein the one or more channels ensure passage of sample, reagents, and fluid between the sample inlet, the reagent inlet, and the outlet.

The prepared sample produced by the microfluidic system as further described herein can be subsequently analyzed by a machine configured to carry out a method selected from the group consisting of: PCR, TMA, SDA, and NASBA. The prepared sample produced by the microfluidic system may be further processed and analyzed by a variety of target amplification and/or signal amplification techniques and may also be analyzed by restriction digestion followed by capillary electrophoresis and/or mass spectrophotometry analysis, and other examples of techniques commonly referred to as genomic and proteomic technologies.

Preferred embodiments of the microfluidic system further comprise one or more components of computing machinery, such as: a visual display that communicates to a user of the system information including the current status of the system, progress of sample preparation, and a warning message in case of malfunction of either system or cartridge; an interface for connecting the system to a computer or a network of computers; a computer-readable memory which stores instructions for operating the control circuitry; a processing unit for executing the instructions; and an input device for accepting information from a user.

Other preferred embodiments of the system described herein utilize a cartridge that is configured to accept two or more separate samples. Still other preferred embodiments of the system are configured to accept two or more cartridges, preferably three cartridges, any one cartridge of which is configured to accept two or more separate samples.

Also further described herein are embodiments of a microfluidic component for converting a sample containing one or more polynucleotides into a form suitable for analyzing the one or more polynucleotides, the component comprising: a sample inlet for receiving the sample; a reagent inlet for accepting one or more reagents; an outlet for directing prepared sample into a PCR tube; and one or more channels configured to transmit volumes of fluid in the range 0.1-50 μl; wherein the one or more channels ensure passage of sample, reagents, and fluid between the sample inlet, the reagent inlet, and the outlet; and wherein the microfluidic component, in conjunction with an external source of heat, is configured to react the sample and the reagents, in order to produce a prepared sample suitable for analyzing the one or more polynucleotides.

Other embodiments still further include a multi-sample cartridge configured to accept a number of samples, in particular embodiments eight samples, wherein the samples include at least a first sample and a second sample, wherein the first sample and the second sample each contain one or more polynucleotides. The samples can each be converted into respective forms suitable for analyzing the one or more polynucleotides, the multi-sample cartridge comprising: at least a first microfluidic component and a second microfluidic component, separably affixed to one another, wherein each of the first microfluidic component and the second microfluidic component is as previously described herein, and wherein the first microfluidic component accepts the first sample, and wherein the second microfluidic component accepts the second sample. The sample inlets of adjacent cartridges are reasonably spaced apart from one another to prevent any contamination of one sample inlet from another sample when a user introduces a sample into any one cartridge.

In preferred embodiments, the multi-sample cartridge has a size substantially the same as that of a 96-well plate as is customarily used in the art. Advantageously, then, the cartridge may be used with plate handlers used elsewhere in the art. Still more preferably, however, the multi-sample cartridge is designed so that a spacing between the centroids of mounts for PCR tubes is 9 mm, which is an industry-recognized standard. This means that, in certain embodiments the center-to-center distance between nozzles in the cartridge that deliver materials to adjacent PCR tubes, as further described herein, is 9 mm. In still other preferred embodiments, the multi-sample cartridge comprises a first PCR tube attached to the first microfluidic component, and a second PCR tube attached to the second microfluidic component. Each PCR tube is preferably removably affixed to the cartridge.

Additionally described herein are methods, including but not limited to a method of converting a sample comprising a number of cells that contain one or more polynucleotides into a form suitable for analyzing the one or more polynucleotides, the method comprising: introducing from about 0.1-2.0 mL of the sample and an excess quantity of air into a bulk lysis chamber; lysing cells in the sample by applying heat to the bulk lysis chamber, to raise the sample to a first temperature, thereby producing a lysate containing the one or more polynucleotides; capturing one or more polynucleotides in the lysate on an affinity matrix, such as one or more beads; causing the beads to leave the bulk lysis chamber and be trapped on a filter; washing the beads with a wash reagent; displacing the wash reagent with a release buffer; heating the beads to a second temperature, thereby releasing the one or more polynucleotides; and causing the one or more neutralized polynucleotides to be transferred to a PCR tube. In preferred embodiments, the sample is dissolved in one or more lysis reagents in the bulk lysis chamber prior to applying heat to it. In other preferred embodiments, after heating the beads to the second temperature, the method comprises combining a neutralization buffer with the one or more polynucleotides to produce one or more neutralized polynucleotides, which are then transferred to the PCR tube.

Also further described herein are methods that include a method of analyzing a sample comprising a number of cells that contain one or more polynucleotides, the method comprising: converting the sample into a form suitable for analyzing the one or more polynucleotides, using methods as described herein; and analyzing the sample using a method selected from the group consisting of: PCR, TMA, SDA, and NASBA.

Further details of one or more embodiments are set forth in the accompanying drawings, and the description hereinbelow. Other features, objects, and advantages thereof will be apparent from the description and drawings, and from the claims.

### DESCRIPTION OF DRAWINGS

FIG. 1 shows a perspective view of an exemplary microfluidic system.

FIGS. 2A-2F show exploded views of the exemplary microfluidic system of FIG. 1, and its operation in conjunction with a microfluidic cartridge.

FIGS. 3A, 3B and 3C illustrate plan views of exemplary multi-sample cartridges.

FIG. 4A shows a cross-sectional view of an exemplary microfluidic cartridge as further described herein and a plan view of a microfluidic component of the cartridge.

FIG. 4B shows an exploded view of an exemplary cartridge showing various pieces of its manufacture.

FIG. 5 shows a view of an underside of a microfluidic cartridge, as further described herein.

FIG. 6 shows a view of an exemplary nozzle for dispensing material into a PCR tube, as found on the underside of a microfluidic cartridge, as further described herein.

FIG. 7 shows an exemplary array of heater actuators used in conjunction with a microfluidic cartridge, as further described herein.

FIG. 8 shows part of the array of heater actuators of FIG. 7, in conjunction with part of a microfluidic cartridge, as further described herein.

FIG. 9 shows a region of the part of the array of heater actuators of FIG. 8, in conjunction with part of a microfluidic cartridge, as further described herein.

FIG. 10 shows a plan view of an exemplary microfluidic component as further described herein.

FIG. 11 is a cross-sectional view of an exemplary processing region for retaining polynucleotides and/or separating polynucleotides from inhibitors.

FIG. 12 depicts an exemplary valve.

FIGS. 13A and 13B illustrate an exemplary double valve in respectively open and closed states.

FIG. 14 is a cross-sectional view of an exemplary actuator, and also depicts an exemplary gate.

FIGS. 15-27, describe steps in operation of an exemplary microfluidic cartridge as further described herein.

Like reference symbols in the various drawings indicate like elements.

### DETAILED DESCRIPTION

Analysis of biological samples often includes determining whether one or more polynucleotides (e.g., a DNA, RNA, tRNA, mRNA, or rRNA) is present in the sample. For example, one may analyze a sample to determine whether a polynucleotide indicative of the presence of a particular pathogen is present. As used herein, the terms polynucleotide and nucleic acid compound may be used interchangeably and are taken to mean polymeric organic molecules formed from recurring or non-recurring sequences of one or more of the naturally occurring nucleic acids, adenine, guanine, cytosine, thymine, and uracil.

Typically, biological samples are complex mixtures. For use herein, a sample may be provided as any matrix including but not limited to: a blood sample, a tissue sample (e.g., a swab of, for example, nasal, buccal, anal, or vaginal tissue), a biopsy aspirate, a lysate, as fungi, as bacteria, or as food samples such as are used in testing foodstuffs. Where found in food samples, the foodstuffs can include dairy products such as cheese or milk, and staples such as grain, corn, rice, or maize. Polynucleotides to be determined may be contained within particles (e.g., cells, such as white blood cells and/or red blood cells), tissue fragments, bacteria (e.g., gram positive bacteria and/or gram negative bacteria, fungi, spores). One or more liquids (e.g., water, a buffer, blood, blood plasma, saliva, urine, spinal fluid, or organic solvent) is typically part of the sample and/or is added to the sample during a processing step.

Methods for analyzing biological samples include steps of obtaining a biological sample in a form that can be handled in a laboratory (e.g., in the form of a swab), releasing polynucleotides from particles (e.g., bacteria or other cells) in the sample, amplifying one or more of the released polynucleotides (e.g., by PCR), and determining the presence (or absence) of the amplified polynucleotide(s) (e.g., by fluorescence detection).

Biological samples also typically include inhibitors (e.g., mucosal compounds, hemoglobin, faecal compounds, and DNA binding proteins). Such compounds inhibit attempts to determine the presence of polynucleotides in the sample. For example, such inhibitors can reduce the amplification efficiency of polynucleotides by PCR and other enzymatic techniques for determining the presence of polynucleotides. If the concentration of inhibitors is not reduced relative to the polynucleotides to be determined, the analysis can produce false negative results. Accordingly, preferred methods and related systems for preparing biological samples (e.g., samples having one or more polynucleotides to be determined) reduce the concentration of inhibitors relative to the concentration of polynucleotides to be determined.

System

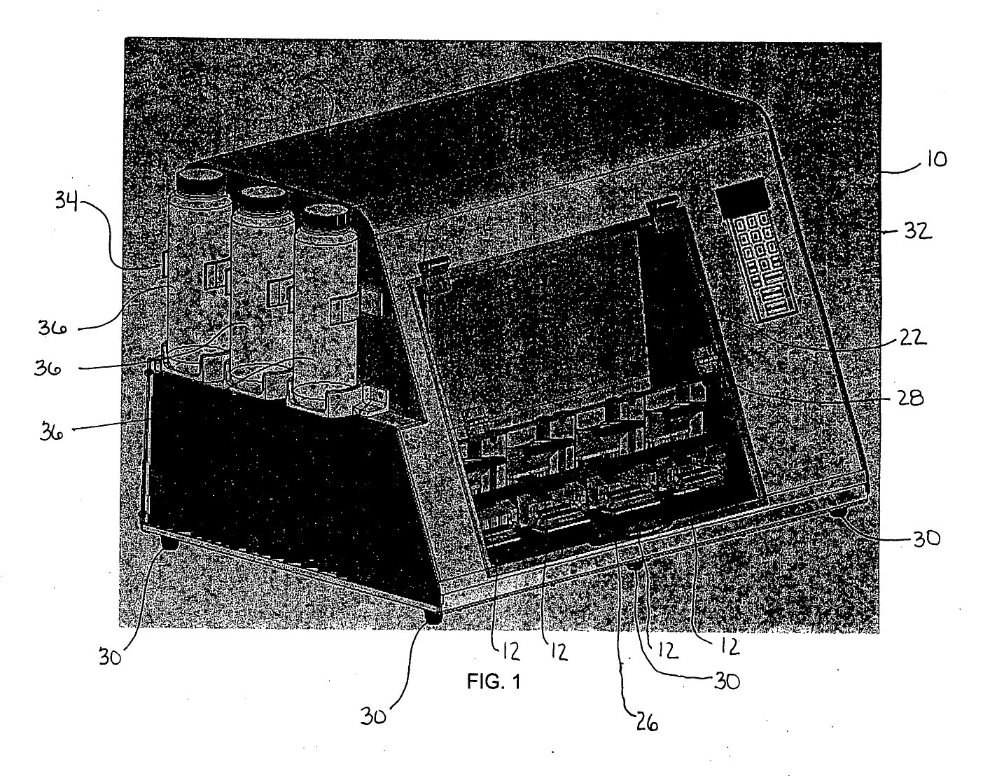

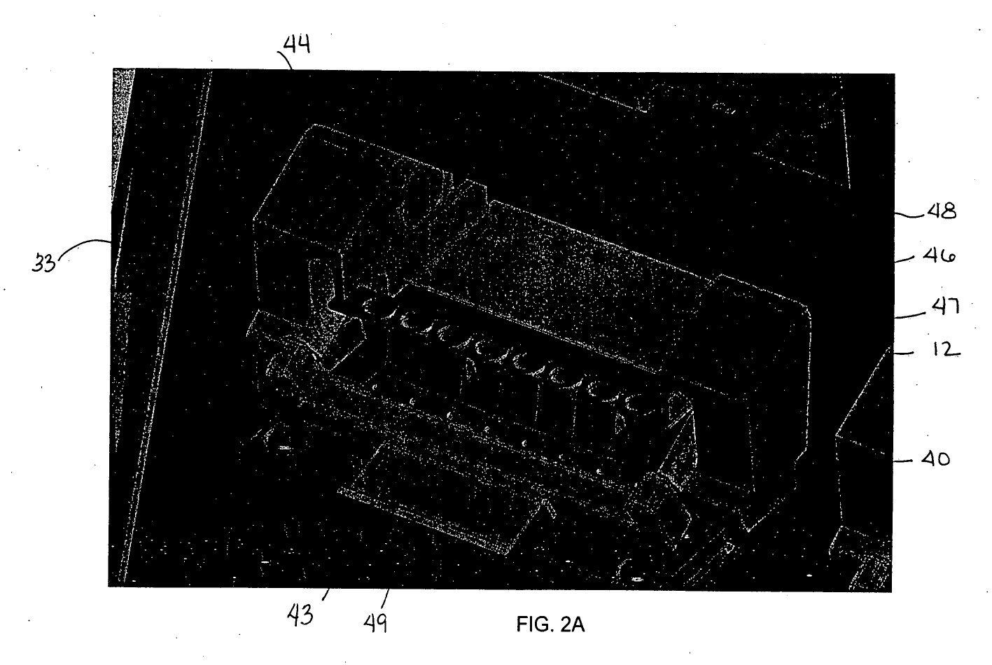

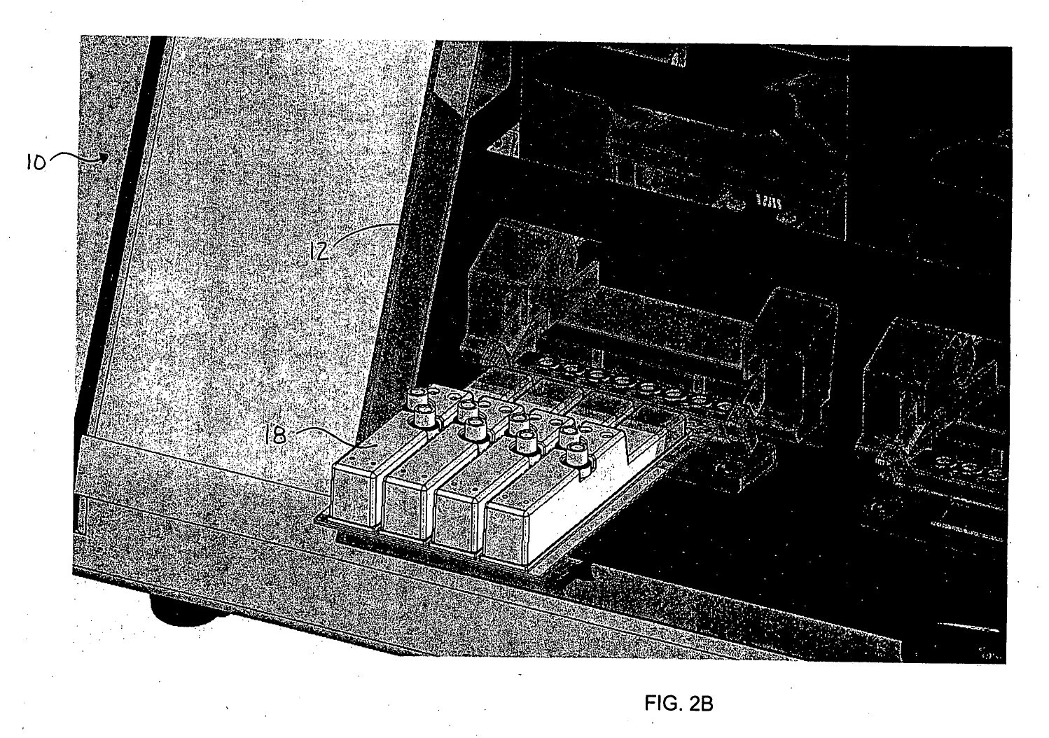

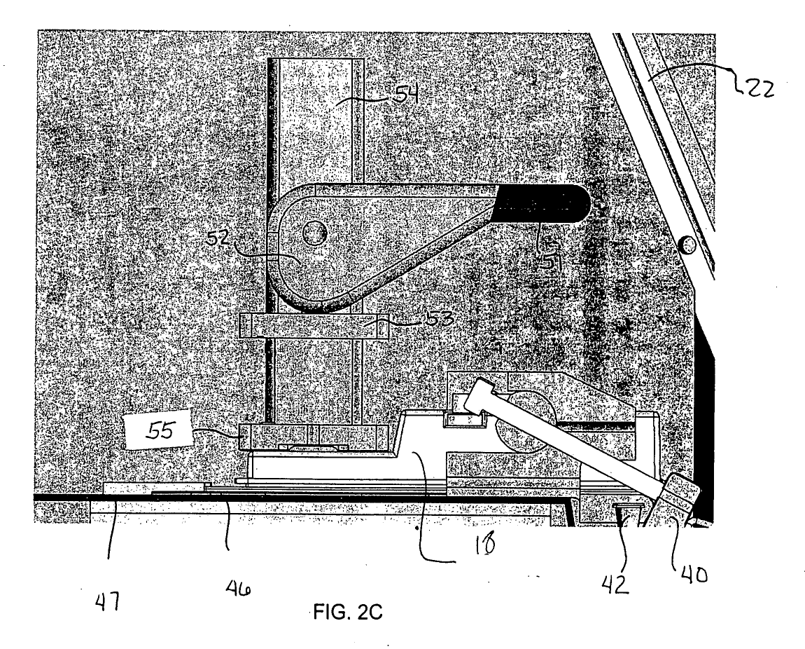

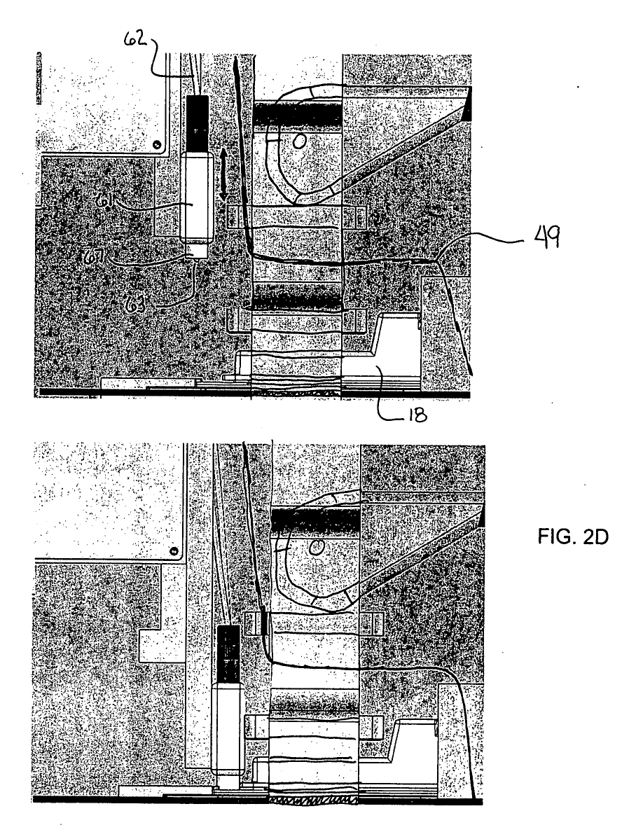

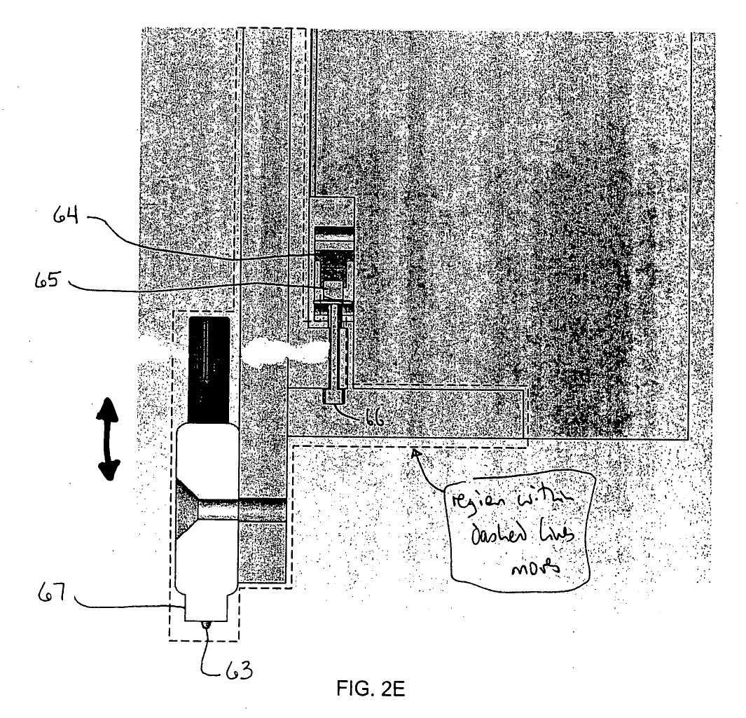

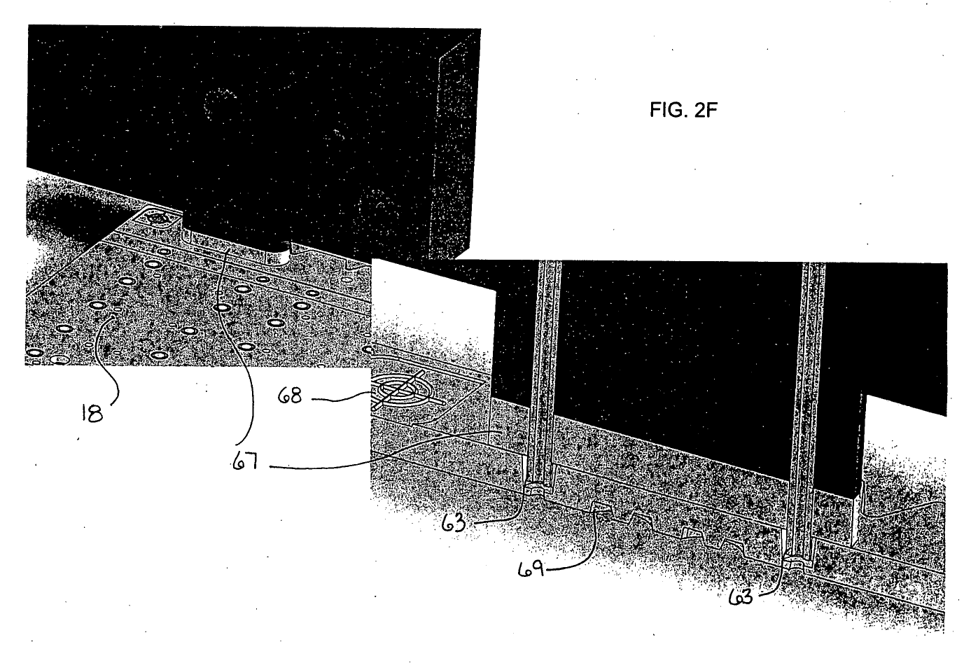

FIG. 1 depicts an exemplary microfluidic system 10 for converting a sample containing one or more polynucleotides into a form suitable for analyzing the one or more polynucleotides, for example according to methods described herein. FIGS. 2A-2F show exploded views of various aspects of exemplary system 10.

Four cartridge receiving elements 12 are depicted in FIG. 1, though it would be understood that other suitable embodiments of device 10 may have more, or fewer, receiving elements, such as but not limited to 1, 2, 3, 6, 8, 10, 12, 16, or 20 receiving elements. System 10 optionally has a closeable door 22, that covers the region of system 10 in which the cartridge receiving elements are situated. Door 22 may be transparent, for example made of Perspex or some similar material, so that a user may monitor visually the system's activity. Cartridge receiving elements 12 independently accept an insertable and removable cartridge such as a microfluidic cartridge as further described herein, and also such as a multi-sample cartridge, as further described herein, wherein a mechanical key (not shown) may facilitate accurate insertion of the cartridge. FIG. 1 shows that the optional door 22 is preferably closed during preparation of a sample. Door 22 is shown hinged at its top edge with one or more hinges 24, but may also be hinged at its lower, or its left, or right edges, consistent with the overall operation of system 10. Door 22 is further depicted with an optional handle 26 for ease of opening and closing. Door 22 is still further depicted in FIG. 1 with an optionally hingeable middle section, as accomplished by one or more hinges 28. Such an optionally hingeable middle section facilitates partial opening of the door, as well as to create a more manageable folded configuration of the door when open.

System 10 also preferably comprises an area 35 for storing reagents. Such an area may be located within housing 33 of system 10 but may also be on the outer surface of housing 33, as depicted in FIG. 1. Depicted in FIG. 1 are three reagent bottles 36 mounted externally to housing 33 via one or more mounting brackets 34. Reagent bottles 36 contain, respectively, release buffer, wash buffer, and neutralization buffer, and are configured to deliver the respective reagents to the samples during sample preparation. The external mounting of reagent bottles 36 advantageously permits a user to readily see when any one or more bottle requires re-filling. The incorporation of reagent bottles into system 10 is advantageous because it permits system 10 to be easily transportable from one location to another within a laboratory, without need for disconnecting and reconnecting delivery tubes from external reagent storage to the system. In other embodiments, however, where it is desired to operate system 10 for long periods of time without frequent user intervention to refill reagent bottles, the reagents may be supplied from larger containers, not attached to or contained inside system 10, but situated elsewhere and configured to deliver reagents to system 10 via one or more tubes, supply lines, or pipes.

System 10 also may comprise one or more stabilizing feet 30 that cause the housing 33 to be elevated above a surface on which system 10 is disposed, thereby permitting ventilation underneath system 10, and also providing a user with an improved ability to lift system 10. There may be 2, 3, 4, 5, or 6, or more feet 30, depending upon the size of system 10. Feet 30 are preferably made of rubber, or plastic, or metal, and elevate housing 33 of system 10 by from about 2 to about 10 mm above a surface on which it is situated.

Microfluidic system 10 further optionally comprises a display 20 that communicates information to a user of the system. Such information includes but is not limited to: the current status of the system; progress of sample preparation; and a warning message in case of malfunction of either system or cartridge. Display 20 is preferably used in conjunction with an input device 32, through which a user may communicate instructions to system 10. Input device may be a touch-screen, a key-pad, or a card-reader. Input device 32 may further comprise a reader of formatted electronic media, such as, but not limited to, a flash memory card, memory stick, USB-stick, CD, or floppy diskette. Input device 32 may further comprise a security feature such as a fingerprint reader, retinal scanner, magnetic strip reader, or bar-code reader, for ensuring that a user of system 10 is in fact authorized to do so, according to pre-loaded identifying characteristics of authorized users. Input device 32 may be additionally linked to an external input device (not shown in FIG. 1) such as a computer keyboard, or a computer mouse, for accepting a user's instructions. Input device 32 may additionally—and simultaneously—function as an output device for writing data in connection with sample analysis. For example, if input device 32 is a reader of formatted electronic media, it may also be a writer of such media. Data that may be written to such media by device 32 includes, but is not limited to, environmental information, such as temperature or humidity, pertaining to an analysis, as well as a diagnostic result, and identifying data for the sample in question.

System 10 preferably includes microprocessor circuitry, in communication with input device 32 and display 20, that accepts a user's instructions and controls analysis of samples. System 10 may further include a computer network connection that permits extraction of data to a remote location, such as a personal computer, personal digital assistant, or network storage device such as computer server or disk farm. The computer network connection may be wireless, or may utilize, e.g., ethernet, firewire, or USB connectivity. System 10 may also be connected to a printer, either directly through a directly dedicated printer cable, or wirelessly, or via a network connection. System 10 may further be configured to permit a user to e-mail results of an analysis directly to some other party, such as a healthcare provider, or a diagnostic facility, or a patient.

FIG. 2A shows an exploded view of an exemplary cartridge receiving element 12, from system 10. In this. embodiment, receiving element 12 is configured to accept a multi-sample cartridge having eight sample lanes. Eight PCR tubes 42 may contain reagents for reacting separately with samples in each of the lanes of the cartridge. Such reagents are typically lyophilized reagents such as PCR enzymes, probes and/or primers. Such reagents can experience significant degradation if exposed to temperatures such as room temperature or above and therefore PCR tubes 42 are preferably kept cool in order to prolong reagent lifetime. A preferable manner by which to keep such tubes cool is with a Peltier device (not shown in FIG. 2A). PCR tubes 42 are preferably attached to a PCR-strip 44 for ease of collective mounting. PCR tubes 42 are also shown situated above a shelf having a number of depressions 43 configured to accept the PCR tubes. The depressions 43 can be situated within a cooling device, such as Peltier cooler, to keep the PCR tubes cool when the tubes are sitting in the depressions. In some embodiments the depressions are holes that are deep enough to accept the PCR tubes as deep as their rims.

The remainder of the cartridge receiving element is now described in conjunction with FIG. 2B, which illustrates a way of inserting a multi-sample cartridge 18 into a cartridge receiving element 12 of system 10. Insertable cartridge 18 comprises at least one microfluidic component that, when inserted into receiving element 12, in conjunction with a heating element and control circuitry, is configured to accept one or more polynucleotide containing samples and one or more reagents, and to react the sample and the reagents, in order to produce a prepared sample, delivered to the one or more PCR tubes and in a form suitable for subsequent analysis of the one or more polynucleotides therein. Features of cartridge 18 are further described elsewhere herein.

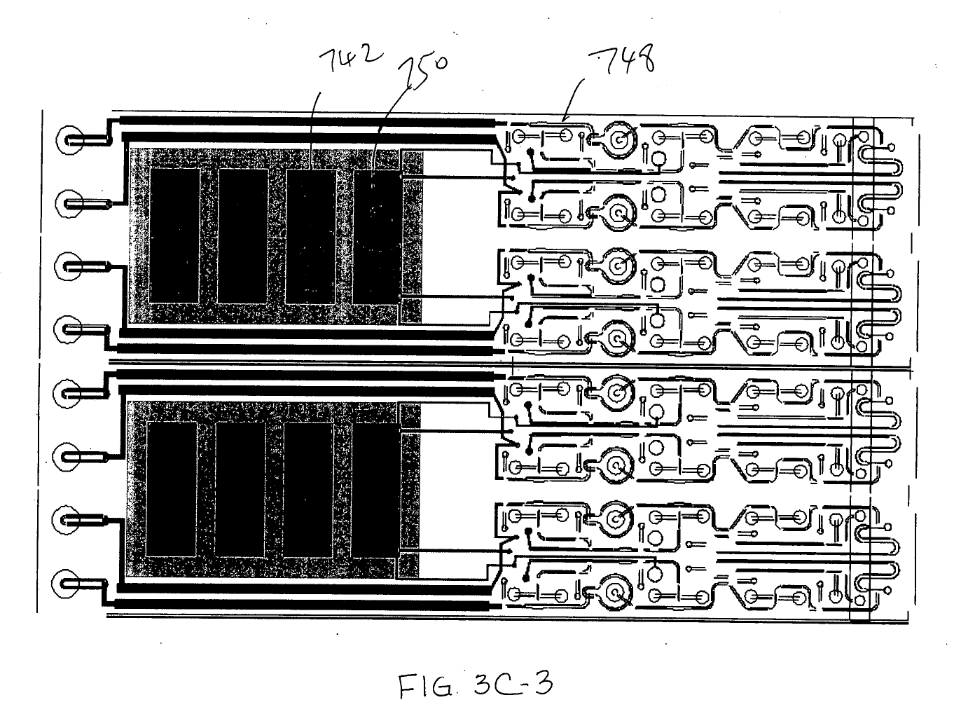

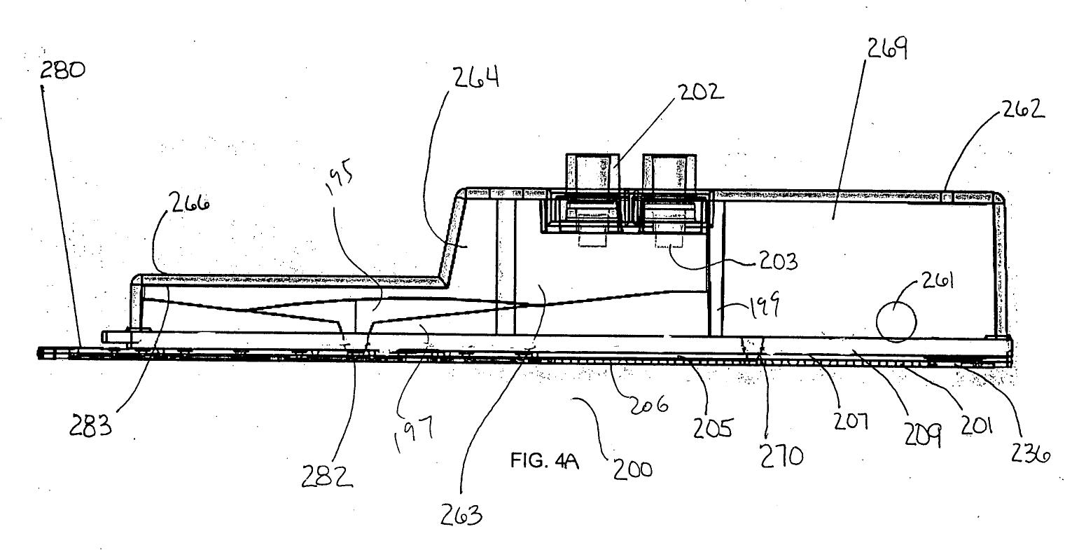

Cartridge receiving element 12 preferably includes a way of ensuring effective registration of the cartridge, via a registration mechanism. A mechanical key on the cartridge, as further described herein, facilitates registration and may be used in conjunction with one or more other mechanical features. Adjustable lever 40, in FIGS. 2A and 2B, is a way of ensuring that a cartridge makes a firm contact in a cartridge receiving element. Although there are many configurations of a lever that can achieve such a contact, it is envisaged that in the embodiment shown in FIGS. 2A and 2B, the cartridge is inserted horizontally into the cartridge receiving element in the direction of the arrow shown, pushed back into the receiving element in order to engage a mechanical key, and then lever 40 is raised underneath the cartridge in a manner that supports the cartridge. Lever 40 may pivot on a cam to provide additional rigidity when engaged with the cartridge. Shelf 49 attached to lever 40 may provide additional support for the cartridge in the embodiment shown in FIG. 2A. Other registration mechanisms may be contemplated, such as utilizing one or more clips, a magnetic attraction, a recessed cavity in which to situate the cartridge, and a snap-fit piece to which the cartridge becomes reversibly fixed, such as by a twisting motion, the locking of the cartridge achieved by a slight deformation of one or more male fittings, e.g., one or more flexible protrusions of either the cartridge or the receiving element, when inserted into one or more complementary female fittings. In other embodiments, the cartridge is positioned at an angle to the horizontal, such as 10° with respect to horizontal, to facilitate flow of sample from the lysis chamber into a microfluidic component of the cartridge. In such embodiments, it is less important to deploy a funnel structure with ramps such as 197 in the lysis chamber, as further described herein with respect to FIGS. 4A and 4B.

Cartridge receiving element also preferably includes a heat source capable of delivering controllable and localized heat to selected portions of the cartridge. Platform 46 in FIGS. 2A and 2B is an area having a plurality of thermal actuators, on which the cartridge rests during analysis, and which is in thermal communication with the cartridge. The plurality of thermal actuators, such as resistive heaters, are configured to heat one or more regions of the cartridge. Microprocessor control circuitry, not shown, is in communication with platform 46 and upon receiving user instructions will cause current to flow to selected thermal actuators to thereby cause one or more regions of the cartridge adjacent to the selected actuators to heat up. In other preferred embodiments, heat source 46 rests on a PC board 47. Thus, together, elements 46 and 47 are but exemplary ways to heat a cartridge.

In another preferred embodiment, a protective barrier 48 shields a user of system 10 from various internal workings and internal components thereof.

FIGS. 2C and 2D show a cross-sectional view of exemplary cartridge receiving element 12, showing various components that facilitate delivery of reagents and heat to cartridge 18. In particular, in order to maintain a good thermal contact between heat source 46 and cartridge 18, one method is to incorporate a user-actuated handle 51 that can apply pressure to cartridge 18. In the embodiment shown in FIG. 2C, handle 51 is attached to a cam-shaft 52 that, when pivoting against fixed ledge 53, causes a plunger 54 to be depressed and platform 55 attached to the plunger to press down against cartridge 18. Platform 55, in the embodiment shown, has a contact heat source that can cause heat to be applied to liquid sample in a lysis chamber of cartridge 18, as further described herein. The pressure exerted on platform 55 not only makes good contact between a heat source in platform 55 and upper surface of cartridge 18, but also causes a good thermal contact between heat source 46 and the microfluidic component on the underside of cartridge 18. Action of cam and plunger 54 also serves to ensure that the position of cartridge 18 is stable during processing. In preferred embodiments, a sensor in communication with platform 55 causes microadjustments of plunger 54 so that undue pressure, such as pressure that would cause undue strain, stress, or damage, is not applied to cartridge 18. One of ordinary skill in the art would understand that a cam and plunger assembly is not the only mechanical arrangement that can apply pressure to cartridge 18 for the purpose of making good thermal contact. For example, a press can be envisaged that utilizes an adjustable screw for changing the height of the press above the cartridge, as can other arrangements that comprise levers and similar mechanisms.

In preferred embodiments, each cartridge receiving area in system 10 has its own independently controllable mechanism for applying pressure to areas of the microfluidic cartridge that are contact heated. Thus, in FIG. 1, each handle above each cartridge receiving area can be depressed by a user independently of the others. Other aspects of the cartridge receiving area not apparent from FIGS. 2A-2C include the use of a platform underneath the cartridge to keep it rigid while pressure is applied.

As would be understood by one of ordinary skill in the art, many mechanisms exist for repetitively delivering precise volumes of liquid reagents to a fixed sample. In one embodiment, the mechanism is purely manual and involves a user actively raising and lowering a dispensing head. In preferred embodiments, the dispensing head is under robotic control. In still other embodiments, the dispensing head uses hydraulics.

FIG. 2D shows an exemplary mechanism for delivering reagents to the microfluidic cartridge. A dispensing head 61, under robotic control, and receiving control signals, e.g., from a microprocessor configured to operate the head, under a user's instructions, is in communication with one or more reagent sources. One or more capillaries 62 feed one or more nozzles 63 with, respectively, one or more reagents such as release buffer, wash buffer, and neutralization buffers, where the one or more reagents are preferably stored on the exterior housing of the system 10, as shown in FIG. 1. Dispensing head 61 has a vertical degree of freedom, as indicated by the arrow in FIG. 2D, that permits it to penetrate and withdraw from the cartridge respectively prior to and after delivering reagent. The two panels of FIG. 2D show the nozzle in a position away from the cartridge, and when delivering reagent. Additionally, and preferably, dispensing head 61 has a degree of freedom sideways—perpendicular to the plane of the paper in FIG. 2D—so that the dispensing head can, e.g., deliver reagent to more than one lane or more than one cartridge of a multi-sample cartridge. Additionally, sideways motion may be for the purpose of permitting the dispensing head to visit more than one cartridge location, such as more than one cartridge receiving element.

A reagent dispenser preferably and optionally has a sensing mechanism that prevents it from going down too far and damaging either a nozzle 63 or the cartridge, or both. Many sensing mechanisms are consistent with the practice of the invention and may use, e.g., contact sensing (e.g., by detecting onset of or disruption of an electrical current), magnetic sensing, optical sensing, or by use of a mechanical spacer that stops the dispensing head from travelling too far. As further shown in FIG. 2E, an exemplary sensing mechanism uses an optical interrupter. Such a mechanism is effective at ensuring that a good seal is obtained between the dispensing head and the cartridge, without resulting in damage to either. In this embodiment, a screw 66, flag 65 and optical interruptor 64 mounted on a fixed assembly work in cooperation with the dispensing head. Once the dispenser abuts the microfluidic cartridge, the screw pushes the flag up into the sensing position of the optical interrupter, which provides feedback to the motor that controls the dispensing head, causing it to cease the motion of the head.

As previously described, it is preferable that a nozzle of the dispensing head makes a good contact with a reagent inlet on the microfluidic cartridge. This can be achieved with a number of different approaches known in the art. An exemplary embodiment is shown in FIG. 2F, which can be viewed in conjunction with FIG. 2E. In the left hand panel of FIG. 2F, gasket 67 is shown poised above a pair of adjacent reagent inlets (such as on adjacent lanes) of a microfluidic cartridge 18. Sighting element 68 may facilitate automatic positioning of the gasket. The right hand panel of FIG. 2F shows a cut-away view of gasket 67, in contact with a pair of adjacent reagent inlets of a microfluidic cartridge. The horizontal separation between the reagent inlets may be 1-2 mm. Notches 69 in the underside of the cartridge exemplify a mechanical key used by the cartridge for positioning in the cartridge receiving element. Reagent dispenser tubes, such as capillaries, are shown in cutaway view also, with tips 63 sunk into respective reagent inlets. The configuration shown in the right hand panel of FIG. 2F exemplifies a good seal between dispenser and cartridge, and is desirable for the purpose of avoiding leaks of reagent sample. Leaks are undesirable, because repetitive leaking of reagents within the interior of system 10 can lead to rapid degradation of components through rust, accumulation of mould, and other sources of water-based damage. Leaks are also undesirable because an incorrect (insufficient) quantity of reagent may ultimately be deployed in the microfluidic device, leading to poor sample preparation quality.

Multi-Sample Cartridge

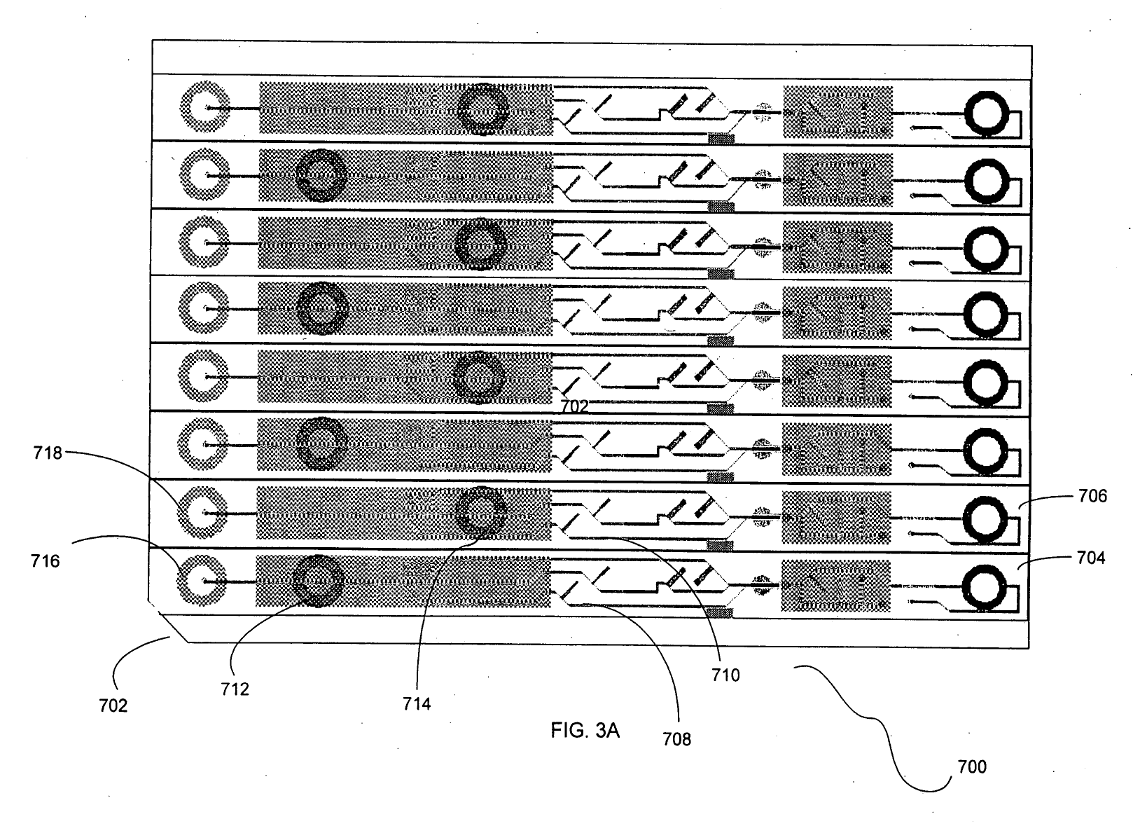

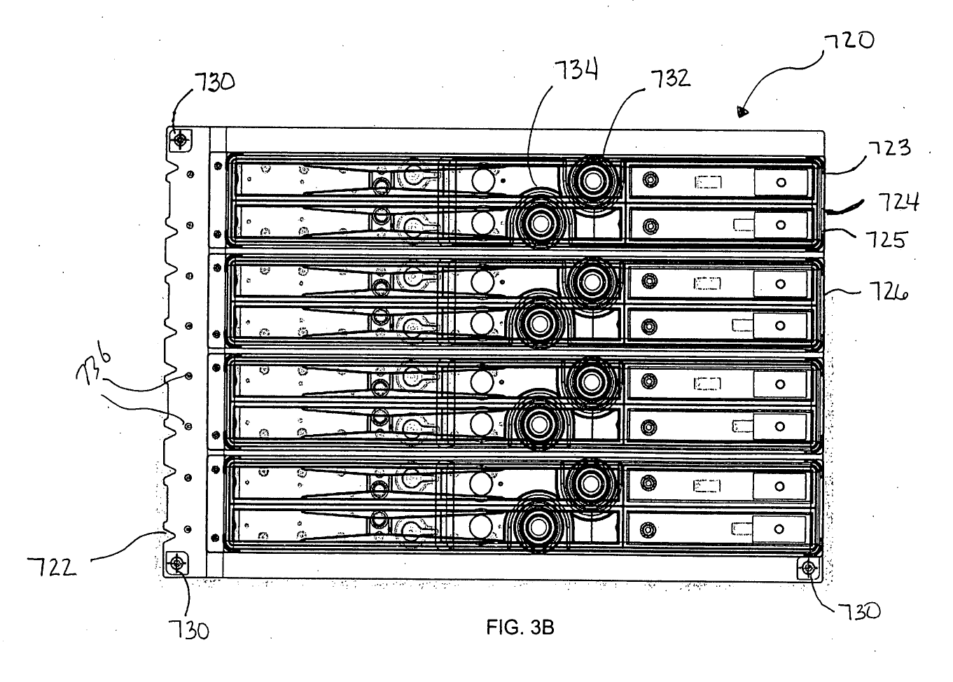

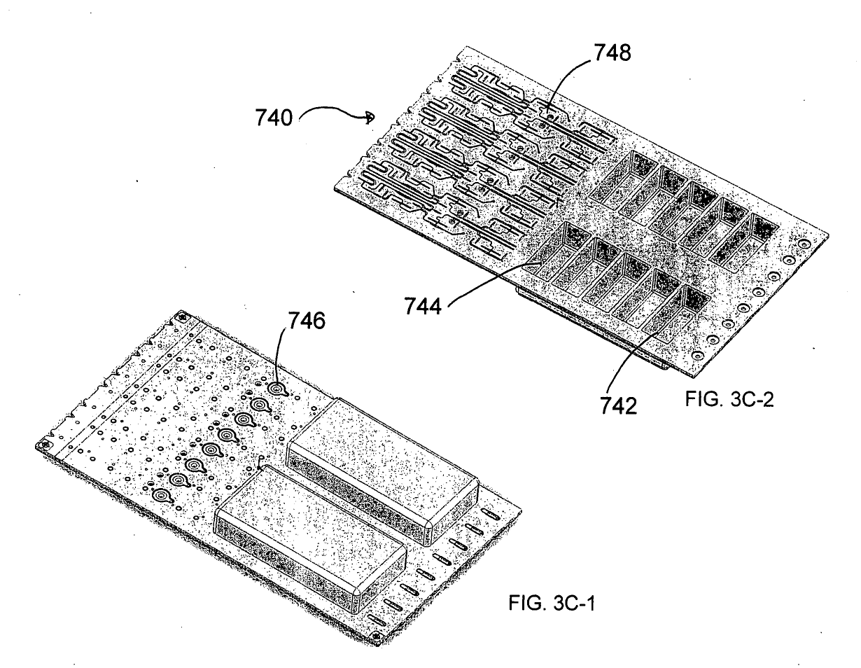

The methods described herein may be practiced with a multi-sample cartridge 700 or 720, as shown in FIGS. 3A, 3B, and 3C respectively. A multi-sample cartridge may be used to convert a number of samples, including at least a first sample and a second sample, wherein the first sample and the second sample each contain one or more polynucleotides (which may be the same as, or different from, one another), into respective forms suitable for analyzing the one or more polynucleotides.

Multi-sample cartridge 700, in which microfluidic circuitry 708, 710 is shown schematically, comprises at least a first microfluidic cartridge 704 and a second microfluidic cartridge 706, separably affixed to one another. Multi-sample cartridge 720 is another embodiment in which sample lanes such as 723 and 725 are grouped in pairs, and comprises at least a first microfluidic cartridge 724 having a first pair of sample lanes, and a second microfluidic cartridge 726 having a second pair of sample lanes, wherein the first and second microfluidic cartridges are separably affixed to one another. A sample lane is an independently controllable set of elements by which a sample can be prepared, according to methods described herein. A lane comprises at least a reagent inlet, a sample luer, a microfluidic component, and a waste chamber, as further described herein in connection with a microfluidic cartridge.

By separably affixed is meant that one cartridge is joined to another such that both can be placed together into a cartridge receiving element of a microfluidic system, but that at least the first and second cartridges could be broken apart from one another and used separately from one another. To facilitate the breaking apart, a score line, for example, may be fabricated between the two cartridges.

In FIG. 3A, preferably each of the first microfluidic cartridge 704 and the second microfluidic cartridge 706 is according to that further described herein, see e.g., FIG. 4, and the first microfluidic cartridge accepts a first sample, and the second microfluidic cartridge accepts a second sample. Thus first cartridge 704 comprises at least a first microfluidic component 708, and second cartridge 706 comprises at least a second microfluidic component 710.

In FIG. 3B, preferably each sample lane of the first microfluidic cartridge 714 and each sample lane of the second microfluidic cartridge 716 is according to that further described herein, see e.g., FIG. 4, and the first microfluidic cartridge accepts a first and second sample, and the second microfluidic cartridge accepts a third and fourth sample. Thus first cartridge 714 comprises at least a first and a second microfluidic component, and second cartridge 716 comprises at least a third and fourth microfluidic component.

Preferably a multi-sample cartridge is the same size as a 96-well plate, as used in the art. Preferably, a multi-sample cartridge has 8 cartridges, as depicted in FIG. 3A, or has 8 lanes, arranged in pairs, as depicted in FIG. 3B. It would be understood that alternative multi-sample cartridges, having different numbers of independent cartridges and/or lanes, are consistent with the methods and apparatus described herein; such numbers include 4, 6, 10, 12, or 16 single-lane cartridges, and 2, 3, 5, 6, or 8 two-lane cartridges. It is additionally possible that a cartridge can be configured with 4, 6, or 8 lanes and be consistent with the description herein.

Still further preferably, each cartridge of a multi-lane cartridge is configured with a PCR tube for each cartridge, separated from one another by 9 mm, or about 9 mm, centroid-to-centroid, and preferably the individual PCR tubes are connected to one another by a strip so that all the tubes can be removed from the multi-lane cartridge simultaneously.

The multi-sample cartridge has additionally and optionally a mechanical key that prevents a user from inserting it into a microfluidic system incorrectly, and also ensures accurate engagement of the cartridge with instrumentation such as a cartridge receiving element 12 of system 10 of FIG. 1. Preferably the mechanical key is engineered on the edge of cartridge 700, or of cartridge 720, that is inserted first into system 10. Such a key can comprise, e.g., a single cut-out corner 702 of the multi-sample cartridge as in FIG. 3A, or several, such as two or more, notches 722 cut in the edge of cartridge 720 of FIG. 3B. Where the key comprises one or more notches, it is preferable that there is at least one notch associated with each lane, as in FIG. 3B, or of each cartridge.

Multi-sample cartridges 700 and 720 have, respectively, one or more luers for sample introduction. In FIG. 3A, there is a luer 712 and a luer 714 associated with, respectively, first cartridge and second cartridge. In FIG. 3B, luers 732 and 734 are associated with, respectively, first lane 723 and second lane 725. In FIGS. 3A and 3B, luers in adjacent cartridges or lanes, are offset with respect to one another. Such an offset is a design feature in one embodiment and facilitates efficient configuration of microfluidic circuitry, but is not a requirement of the multi-sample cartridge.

Multi-sample cartridge 700 also has a first reagent inlet 716 and a second reagent inlet 718 for each of first cartridge 704 and second cartridge 706, respectively. Multi-sample cartridge 720 also has a reagent inlet 736 associated with each sample lane.

Multi-sample cartridge 720 additionally and optionally comprises one or more sighting elements 730 that facilitate positioning of a liquid reagent dispenser head when dispensing reagents into the cartridge. Such sighting elements may be used in conjunction with an optical positioning system used in conjunction with a dispenser head, and as may be incorporated into system 10 by one of ordinary skill in the art.

FIG. 3C, comprising FIGS. 3C-1, 3C-2 and 3C-3, shows an alternative embodiment 740 of a multi-sample cartridge in which multiple lanes (eight are shown) are fabricated in a single microfluidic substrate. It is preferred, in this embodiment, that the chambers and substrate are also integral. In the embodiment shown chambers are arranged in two pairs of rows, such that there are four sample lysis chambers 742, with separate inlets, and one waste chamber 744 per row. Each row of chambers can therefore service four sample lanes. Preferably this cartridge does not have ramped funnels within each lysis chamber (as further described herein) and is therefore inclined at an angle to the horizontal during analysis. FIG. 3C-1 is a perspective view of the cartridge from above showing reagent inlets 746 (the lysis and sample chambers are obscured by a protective cover, and the individual sample inlets are not shown). FIG. 3C-2 is a perspective view from the underside showing schematic microfluidic networks 748. FIG. 3C-3 shows a plan view from the top of the cartridge, showing that each sample inlet 750 and sample chamber communicates with a separate microfluidic network. Other aspects of multi-sample microfluidic cartridges, such as communication with thermal actuator networks, may be accomplished for the example of FIG. 3C as further described herein, as would be understood by one of ordinary skill in the art. The embodiment of FIG. 3C may also utilize a mechanical key such as shown and described with respect to embodiments in FIGS. 3A and 3B.

Microfluidic Cartridge

Microfluidic cartridges as described herein, may adopt a number of different configurations of components without deviating from the spirit of the methods of analysis as described elsewhere herein. Such cartridges are configured to accept, separately, a sample and reagents, to lyse the sample, introduce the sample into a microfluidic network, and deliver an extract containing polynucleotides to an outlet. For example, an exemplary embodiment is found depicted in FIG. 1 of U.S. provisional application Ser. No. 60/726,066, filed Oct. 11, 2005 and incorporated herein by reference.

Referring to FIG. 4, an exemplary multi-sample microfluidic cartridge 200 is shown in cross-section. The following description pertains to a single cartridge or lane as found in the multi-sample cartridge. Cartridge 200 includes first and second layers 205, and 209. First layer 205 functions as a microfluidic substrate and a microfluidic network is found inside. Within first layer there may be a further layer 207, permitting various components of a microfluidic network 201 to be elevated with respect to one another. Second layer 209 is often referred to as a microfluidic substrate because it has one or more holes in it that align with and communicate with vents in the microfluidic substrate. On the exterior surface of the first layer 205 is typically a protective laminate coating 206.

Microfluidic component 201 is configured to accept and to prepare a sample containing one or more polynucleotides. Cartridge 200 typically prepares a sample by lysing cells within the sample, and releasing the one or more polynucleotides in a form suitable for subsequent analysis. Cartridge 200 may also increase the concentration of one or more polynucleotides and/or reduce the concentration of inhibitors relative to the concentration of the one or more polynucleotides in the sample.

Microfluidic cartridge 200 can be fabricated as desired, preferably by injection moulding. Typically, layers 205, 207, and 209 are formed of a polymeric material. Elements of component 201 are typically formed by molding (e.g., by injection molding) layers 207, 205. Layer 206 is typically a flexible polymeric material (e.g., a laminate) that is secured (e.g., adhesively and/or thermally) to layer 205 to seal elements of component 201. Layers 205 and 209 may be secured to one another using adhesive.

Exemplary cartridge 200 also comprises a bulk lysis chamber 264 and a waste chamber a 269. Preferably these two chambers are fabricated as a single piece, and separated by a barrier 199. FIG. 4B shows an exemplary exploded view of cartridge 200 with various of its components as typically fabricated. Interior funnels 197 are optional and have ramped surfaces that cause liquid to flow downwards under force of gravity towards exit hole 282. Side walls 195 of the funnels are optional and facilitate certain fabrication processes.

Cartridge 200 further comprises a sample inlet 202 by which sample material, preferably in the form of a liquid solution containing cells, can be introduced into bulk lysis chamber 264. Two luers are shown, offset with respect to one another, and situated on adjacent cartridges or lanes of multi-sample cartridge 200, in FIG. 4. Preferably, sample inlet 202 takes the form of a luer having a one-way valve 203. The sample inlet directs sample into bulk lysis chamber 264, in which cells in the sample are lysed, when in contact with bulk lysis reagent pellets (not shown) in chamber 264, or by application of heat to chamber 264, or by a combination of both application of heat and contact with reagent pellets. Sample inlet 202 preferably includes a one-way valve that permits material (e.g., sample material and gas) to enter chamber 264 but limits (e.g., prevents) material from exiting chamber 264 by the sample inlet. Typically, the inlet includes a fitting (e.g., a luer fitting) configured to mate with a sample input device (e.g., a syringe) to form a gas-tight seal. Lysis chamber 264 typically has a volume of about 5 milliliters or less (e.g., about 4 milliliters or less). Prior to use, lysis chamber 264 is typically filled with a gas (e.g., compressed air 263).

In general, the volume of sample introduced is smaller than the total volume of lysing chamber 264. For example, the volume of sample may be about 50% or less (e.g., about 35% or less, about 30% or less) of the total volume of chamber 264. A typical sample has a volume of about 3 milliliters or less (e.g., about 2.0 milliliters or less, or about 1.5 milliliters or less). A volume of gas (e.g., air) is generally introduced to chamber 264 along with the sample. Typically, the volume of gas introduced is about 50% or less (e.g., about 35% or less, about 30% or less) of the total volume of chamber 264. The volume of sample and gas combine to pressurize the gas already present within chamber 264.

Bulk lysis reagent pellets when used preferably contain one or more particles such as DNA capture beads (not shown) that are designed to retain polynucleotide molecules. Particles are preferably modified with at least one ligand that retains polynucleotides (e.g., preferentially as compared to inhibitors). Exemplary ligands for preferentially retaining polynucleotides include, for example, polyamides (e.g., poly-cationic polyamides such as poly-L-lysine, poly-D-lysine, poly-DL-ornithine, and poly-ethylene-imine, polyhistidine). Ligands such as polyboronic acid can also be used for retaining RNA. Other ligands include, for example, intercalators, poly-intercalators, minor groove binders, polyamines (e.g., spermidine), homopolymers and copolymers comprising a plurality of amino acids, and combinations thereof. In some embodiments, the ligands have an average molecular weight of at least about 5,000 Da (e.g., at least about 7,500 Da, or at least about 15,000 Da). In some embodiments, the ligands have an average molecular weight of about 50,000 Da or less (e.g., about 35,000, or less, about 27,500 Da or less). In some embodiments, the ligand is a poly-lysine ligand attached to the particle surface by an amide bond.

In certain embodiments, the ligands are resistant to enzymatic degradation, such as degradation by protease enzymes (e.g., mixtures of endo- and exo-proteases such as pronase) that cleave peptide bonds. Exemplary protease resistant ligands include, for example, poly-D-lysine and other ligands that are enantiomers of ligands susceptible to enzymatic attack.

Particles for retaining polynucleotides are typically formed of a material to which the ligands can be associated. Exemplary materials from which particles can be formed include polymeric materials that can be modified to attach a ligand. Typical polymeric materials provide or can be modified to provide carboxylic groups and/or amino groups available to attach ligands. Exemplary polymeric materials include, for example, polystyrene, latex polymers (e.g., polycarboxylate coated latex), polyacrylamide, polyethylene oxide, and derivatives thereof. Polymeric materials that can be used to form particles 218 are described in U.S. Pat. No. 6,235,313 to Mathiowitz et al., which is incorporated herein by reference. Other materials include glass, silica, agarose, and amino-propyl-tri-ethoxy-silane (APES) modified materials.

Exemplary particles that can be modified with suitable ligands include carboxylate particles (e.g., carboxylate modified magnetic beads, such as Sera-Mag Magnetic Carboxylate modified beads, Part #3008050250, Seradyn, and Polybead carboxylate modified microspheres, available from Polyscience, catalog no. 09850). In some embodiments, the ligands include poly-D-lysine and the beads comprise a polymer (e.g., polycarboxylate coated latex).

In general, the ratio of mass of particles to the mass of polynucleotides retained by the particles is no more than about 25 or more (e.g., no more than about 20, no more than about 10). For example, in some embodiments, about 1 gram of particles retains about 100 milligrams of polynucleotides.

The particles typically have an average diameter of about 20 microns or less (e.g., about 15 microns or less, about 10 microns or less). In some embodiments, particles 218 have an average diameter of at least about 4 microns (e.g., at least about 6 microns, at least about 8 microns).

The density of particles 218 in the lysis pellets is typically at least about 10⁸ particles per milliliter (e.g., about 10⁹ particles per milliliter).

In some embodiments, at least some (e.g., all) of the particles are magnetic. In alternative embodiments, few (e.g., none) of the particles are magnetic.

In some embodiments, at least some (e.g., all) of the particles are solid. In some embodiments, at least some (e.g., all) of the particles are porous (e.g., the particles may have channels extending at least partially within them).

In an embodiment in which heat is applied to the sample in bulk lysis chamber 264, the volume of sample in chamber 264 is such that the upper level of the liquid is in contact with the inside surface 283 of an area 266 of chamber 264. Area 266 is preferably flat and is configured to receive heat from a heat source, whereby the heat effectuates lysis of the cells in the liquid sample. Preferably the heating is by contact heating and preferably it causes the sample to reach a temperature of between 55 and 85° C., and still more preferably between 65 and 75° C. It is noted that the material from which the cartridge is made is typically a good insulator and therefore the outside of the cartridge may have to reach a temperature of 20-40° C., e.g., 30° C., in excess of the desired temperature of the sample.

After the sample has been lysed in lysis chamber 264, the lysed sample flows through outlet 282 into microfluidic network 201.

Cartridge 200 still further comprises a reagent inlet 280 in communication with microfluidic network 201. Typically reagent inlet 280 is of the form of a pierceable inlet, such as a septum. Reagent inlet 280 may also be configured to make a tight seal with a nozzle of a reagent delivery head, as further described herein in connection with system 10.

Cartridge 200 further comprises an outlet 236 by which a prepared sample can be removed (e.g., expelled or extracted). Outlet 236 is preferably configured to direct prepared sample into a PCR tube (not shown in FIG. 4) such as are used in the art. Preferably such a PCR tube 237 is detachable from cartridge 200 and is typically one of those used throughout the biotechnology industry, and is thus typically made of a plastic material such as polypropylene, and configured to fit other laboratory equipment such as a thermal cycler for performing PCR, or other equipment for performing analyses such as TMA, SDA, and NASBA. A PCR tube such as is used herein typically has an effective volume of 0.2 ml, though may also have an effective volume of 0.6 ml. Representative PCR tubes for use with the methods and apparatus described herein are available from suppliers that include USP, Inc., San Leandro, Calif. (see http://www.uspinc.com/PCRtubes.htm). Preferably, PCR tubes for use with the present invention are connected to one another in strips of 8 and are used with a multi-sample cartridge as further described herein.

Cartridge 200 also has a waste chamber 269 that receives waste from microfluidic network 201 via inlet hole 270. When liquid from microfluidic component 201 flows into waste chamber 269 via hole 270 and is followed by air expelled through hole 270, the liquid has a tendency to foam, and overflow from vent 262. To reduce this phenomenon, waste chamber 269 may contain one or more tablets of an anti-foaming agent such as, but not limited to, Simeticone. When used, the tablets are typically 1-4 mm in diameter.

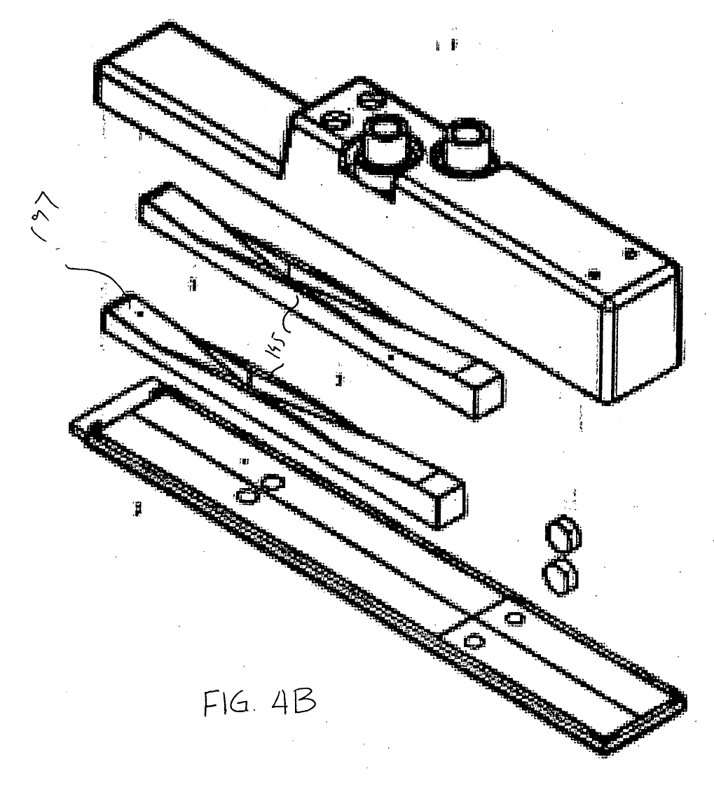

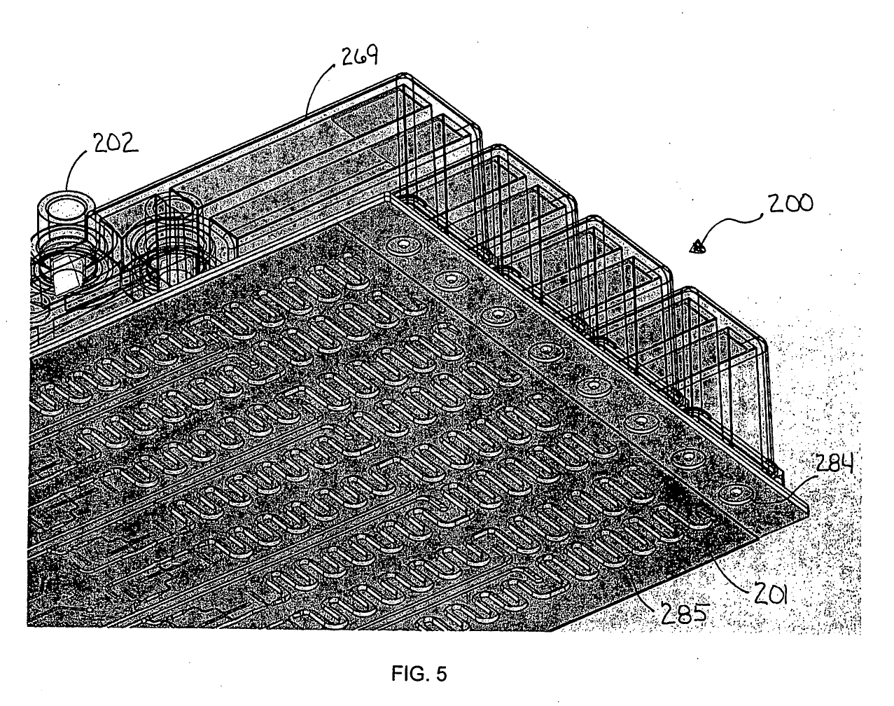

FIG. 5 shows a perspective view of an underside of multi-sample cartridge 200 showing microfluidic component 201 having representative microfluidic channels 285. A nozzle 284 is situated about an outlet 236, and is configured to mate with a top of a PCR tube, to thereby minimize waste during expulsion of polynucleotide containing sample from the microfluidic network into the PCR tube.

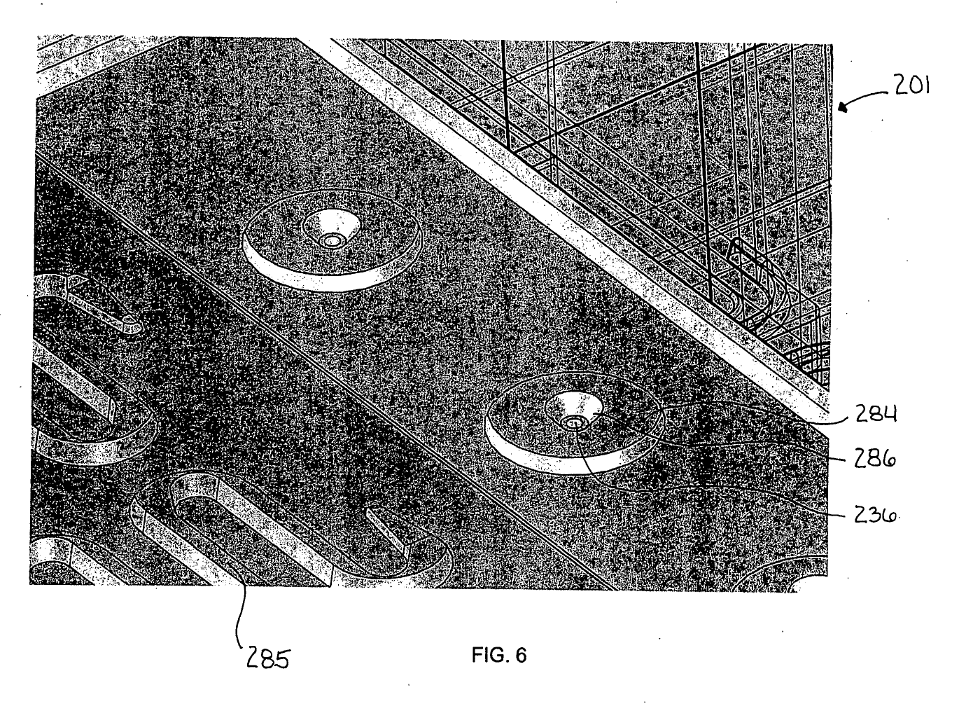

FIG. 6 shows a close-up of an exemplary nozzle 284, showing outlet hole 236 in a raised conical area 286 situated concentrically with respect to the outer rim of nozzle 284. One of ordinary skill in the art would understand that this configuration may be tailored to suit many different shapes and geometries of PCR tube, as used in the art, and is therefore not limited to the configuration depicted in FIG. 6.

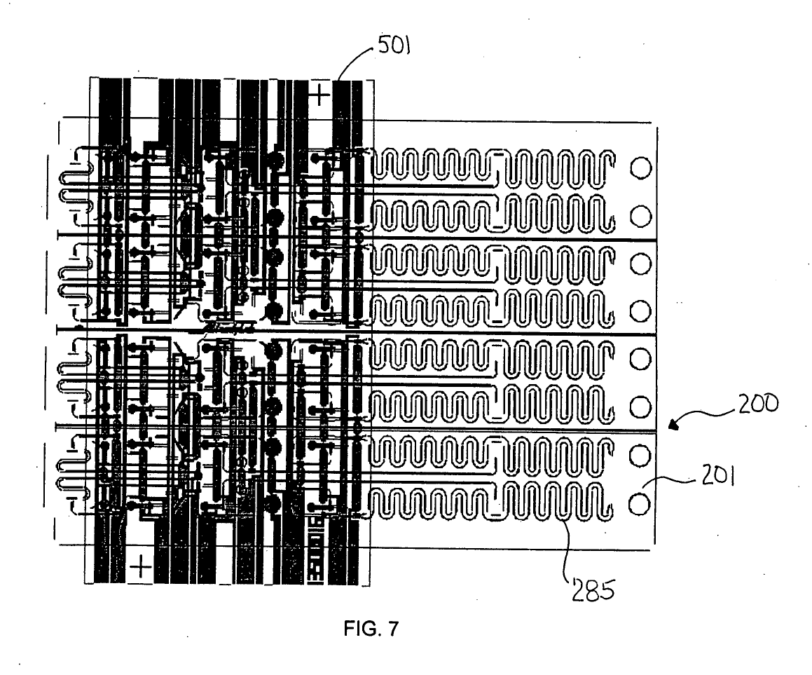

In operation, microfluidic component 201 is situated in close proximity to an array of heaters so that the various elements of the microfluidic component can be controllably and selectively heated. FIG. 7 shows, in overview, a schematic of an array of heaters 501, disposed in a contact heating layer, is disposed in relationship to various microfluidic channels 285 in microfluidic component 201 of microfluidic cartridge 200. Each solid element of array 501 is a conductive element of a heater wafer and is connected, directly or indirectly, to external control circuitry that controls which conductive elements receive current at a particular time. The heater wafer in which heater array 501 is situated is preferably disposed in thermal communication with, such as in contact with, microfluidic component 201. Heater array 501 can be fabricated using design and manufacturing techniques familiar to one of ordinary skill in the art, such as described in PCT/US2005/015345, and U.S. provisional application Nos. 60/567,174, and 60/645,784, all of which are incorporated herein by reference in their entirety.

Heater array 501 can preferably be configured so that individual cartridges or lanes of multi-sample cartridge 200 are heated separately and independently of one another. In other embodiments, heater array 501 is configured so that cartridges or lanes are heated in pairs or in groups, such as 4 lanes at a time in an 8-lane cartridge.

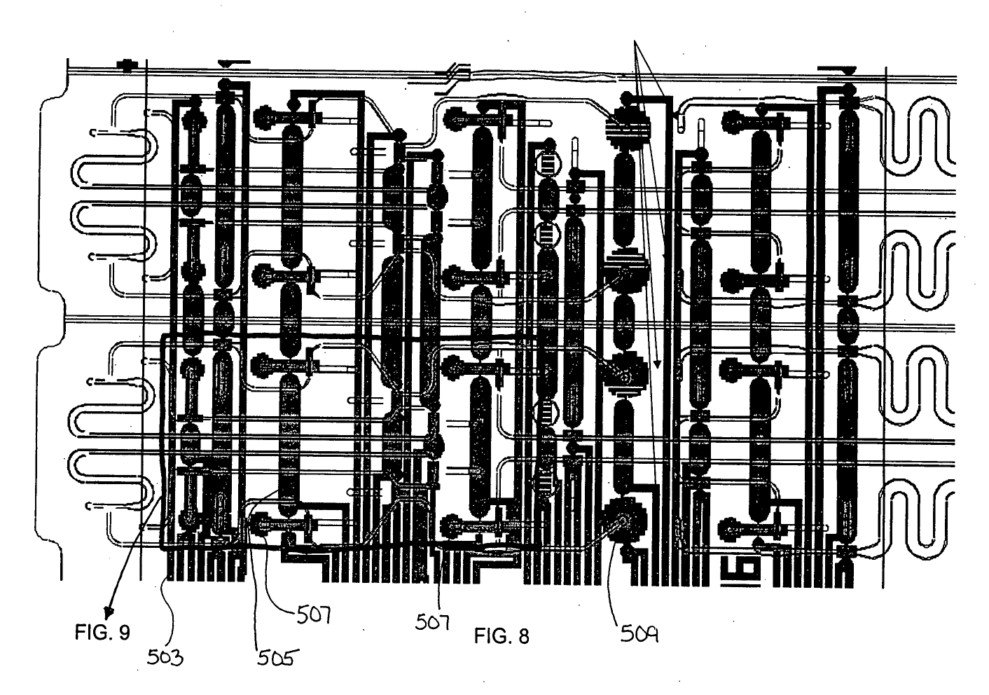

FIG. 8 shows an expanded view of a part of heater array 501 overlayed upon a part of microfluidic network 201. As can be seen in FIG. 8, different parts of heater array 501 have different thicknesses. According to the principle of resistive heating, the thinner parts of array 501 will become the hottest for a given current. Elements such as 505 are current carriers that serve as spacers across regions of microfluidic component 201 that have no microfluidic elements requiring heating. Elements such as 505 thereby generate the least amount of heat of all elements of array 501. Elements 503 achieve an intermediate heating, and are typically of thickness 300 μm, though may range from 280-320 μm, and may also range from 250-350 μm. Elements 507 and 509 achieve the most heating and are disposed directly adjacent microfluidic components such as gates, and valves. Elements 507 and 509 are shown in further detail in FIG. 9.

FIG. 9 shows a further expanded view of a region of FIG. 8, showing structures of elements 507. These structures have fine-scale resistive heaters that generate the greatest amount of heat per unit length of heater array element. The thickness of the wires is typically 40-120 μm, and preferably 50-100 μm, more preferably 60-90 μm, and even more preferably 70-80 μm.

Microfluidic Component

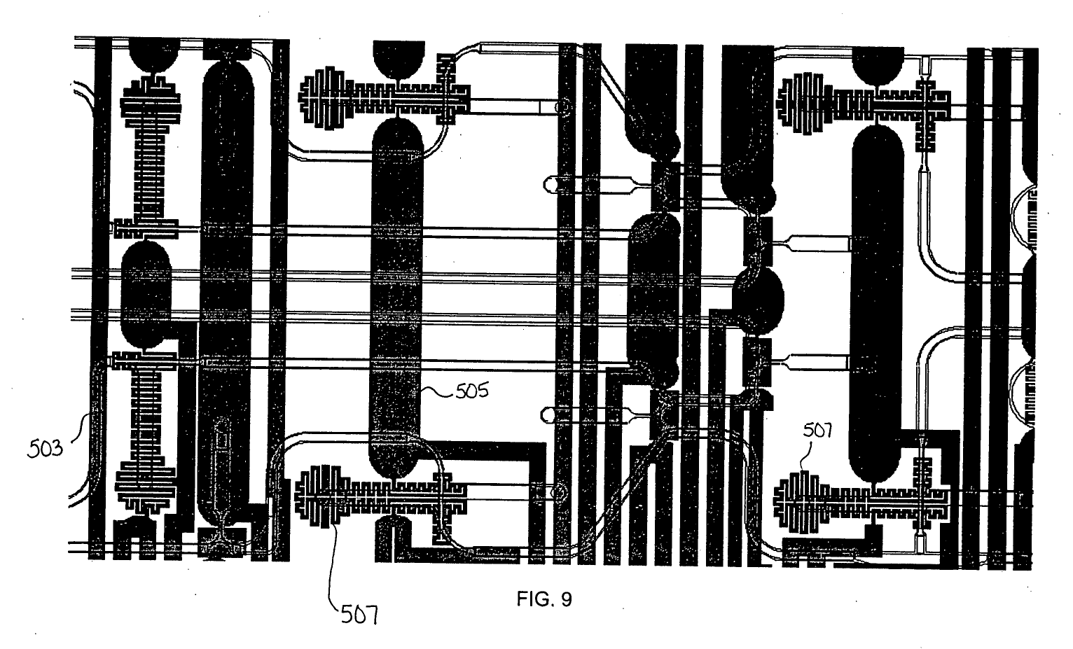

As shown in FIG. 10, microfluidic component 201 typically comprises a number of channels such as channel 234 that are configured to transmit volumes of fluid in the range 0.1-50 μl. Component 201 also preferably comprises one or more microfluidic elements selected from the group consisting of: at least one valve or actuator, at least one gate, at least one hole, at least one vent, at least one filter, and at least one waste chamber. Various configurations of such microfluidic elements are consistent with a microfluidic network that is suitable for practicing methods described herein, and the embodiment shown in FIG. 10 is not intended to be limiting. Accordingly, it would be understood by one of ordinary skill in the art that the configuration of microfluidic elements in FIG. 10 is but one configuration that can be established for practicing the present invention and that other variations of the same are within the scope of the instant invention, although not explicitly found within the instant description. For example, an alternative configuration of microfluidic component is shown in FIG. 2 and described in accompanying text of U.S. provisional application Ser. No. 60/726,066, filed Oct. 11, 2005 and incorporated herein by reference.

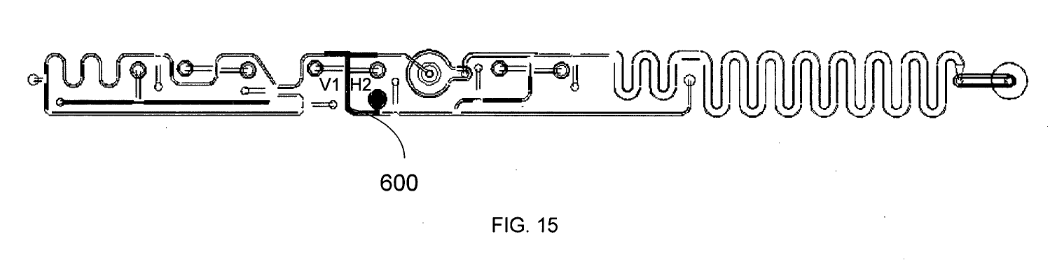

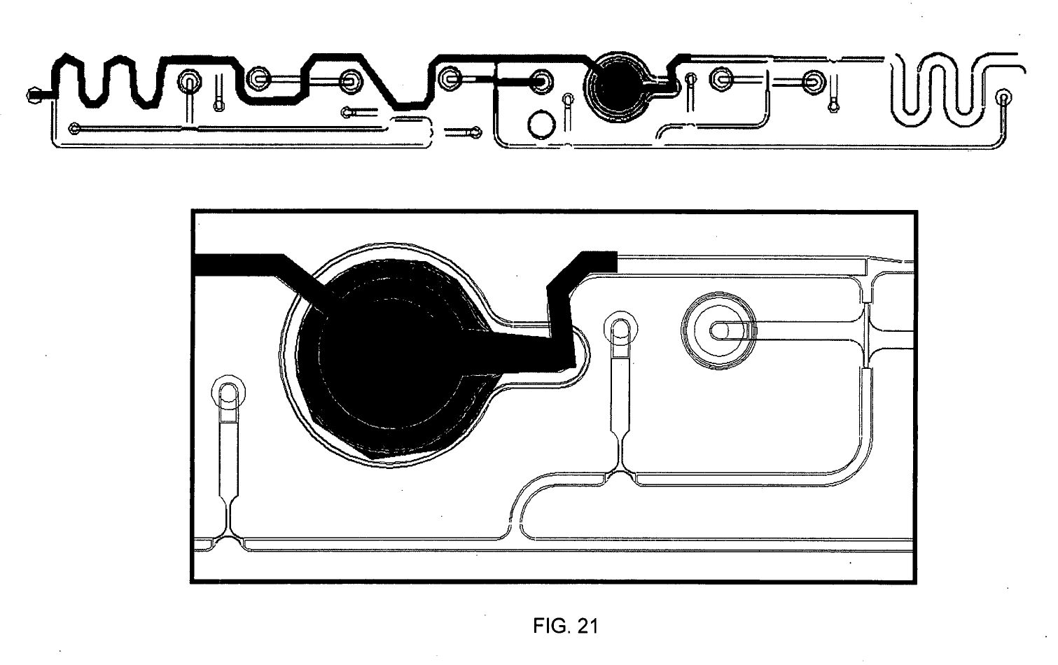

FIG. 10 shows a plan view of component 201, in which various microfluidic elements are labeled as follows: valve (Vi), gate (Gi), hole (Hi), vent (V), and filter (C.), wherein i denotes an integer in the case that there is more than one instance of a particular type of element. In FIG. 10, as with others of FIGS. 15-27, some portions of the microfluidic circuitry are too fine-scale to show up, and gaps are apparent. The exemplary structure that fills such gaps becomes apparent from viewing various panel views in, e.g., FIGS. 21, 22, and 24. The relationship between component 201 and cartridge 200 is at least as follows. Sample inlet 282 is positioned above, though not necessarily directly above, and in communication with hole H2. Reagent inlet 280 is positioned above and in communication with hole H1. Outlet 270 is positioned above and in communication with hole H4. Outlet 236 is positioned above and in communication with hole H3.

Various elements of microfluidic component 201 are substantially defined between layers 207 and 205 but are configured to communicate with layer 209 where applicable.

A channel 204 extends between hole H1 and a gate G5, via gate G4. Channel 206 extends between gate G5 and valve V4. Channels 208 and 211 extend between hole H1 and gate G5, which is also connected to channel 206. Channels 208 and 211 are separated from one another by gate G3 and valve V3. Gate G2 lies on channel 208.

Channel 213 extends from gate G5 to junction 259. Channel 239 extends from junction 259 to filter C. Filter is typically a bead column.

Channel 210 extends from filter C. to junction 215. Gate G6 separates junction 215 from mixing channel 212. Mixing channel 212 extends from gate G6 to hole H3. Thus, in combination, channels 210 and 212 permit filtered sample to travel to hole H3, and thus through a hole 236 via a nozzle 284 such as in FIG. 6 into a PCR tube (not shown). Mixing channel 212 has a capacity to hold between 10 and 50 μl of sample, and can be configured to hold a particular volume within that range by altering the number of turns in the channel.

Channel 234 extends in one direction from hole H2, to junction 259, via valve V1, and in the other direction from hole H2, via gate G2, to hole H4.

Channel 236 extends from junction 257 to junction 215, separated by valve V2 and gate G1.

Various elements of microfluidic component 201 are now described, in turn.

### Filtration Element

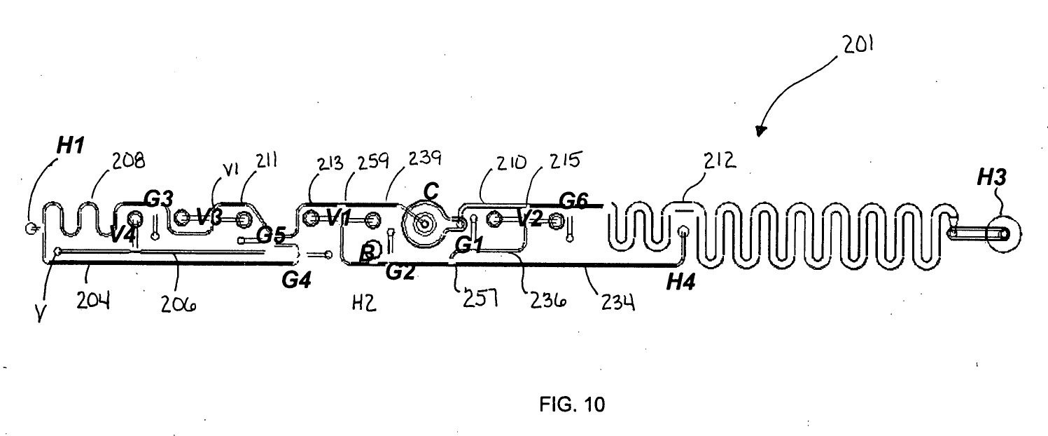

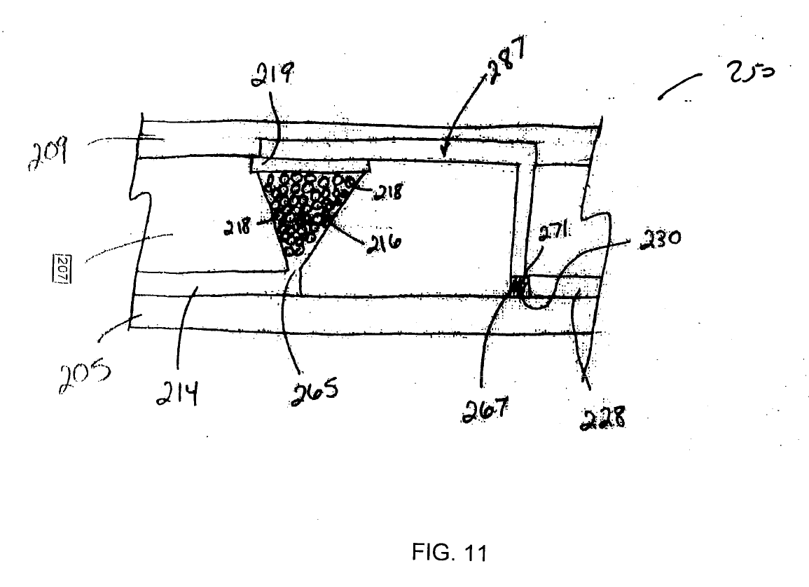

FIG. 11 shows a filtration element 250, such as a bead capture filter or a bead column, for use with a microfluidic component as described herein. Referring to FIG. 3, layers 205, 207, and 209 of microfluidic component 201 are shown. Filtration element 250 retains a plurality of particles 218 (e.g., beads, DNA capture beads, or microparticles such as microspheres) configured to retain polynucleotides of the sample under a first set of conditions (e.g., a first temperature and/or a first pH) and to release the polynucleotides under a second set of conditions (e.g., a second, higher temperature and/or a second, more basic, pH). Typically, the polynucleotides are retained preferentially as compared to inhibitors that may be present in the sample. Particles 218 are confined by a retention member 216 (e.g., a column) through which polynucleotide molecules must pass when moving between the inlet 265 and outlet 267.

Typically, the ligands on the particles 218 retain polynucleotides from liquids having a pH about 9.5 or less (e.g., about 9.0 or less, about 8.75 or less, about 8.5 or less, but preferably more than 7.0). As a sample solution moves through filtration element 250, polynucleotides are retained while the liquid and other solution components (e.g., inhibitors) are less retained (e.g., not retained) and exit the filtration element. In general, the ligands release polynucleotides when the pH is about 10 or greater (e.g., about 10.5 or greater, about 11.0 or greater). Consequently, polynucleotides can be released from the ligand modified particles into the surrounding liquid.

A filter 219, typically made of polycarbonate and typically having a pore size about 1-2 μm smaller than the smallest particles used, prevents particles 218 from passing downstream of the filtration element. A channel 287 connects filter 219 with outlet 267. Filter 219 has a surface area that is larger than the cross-sectional area of inlet 265. For example, in some embodiments, the ratio of the surface area of filter 219 to the cross-sectional area of inlet 265 (which cross-sectional area is typically about the same as the cross-sectional area of channel 214) is at least about 5 (e.g., at least about 10, at least about 20, at least about 50) μm². In some embodiments, the surface area of filter 219 is at least about 1 mm² (e.g., at least about 2 mm², at least about 3 mm²).

In some embodiments, the cross-sectional area of inlet 265 and/or channel 214 is about 0.25 mm² or less (e.g., about 0.2 mm² or less, about 0.15 mm² or less, about 0.1 mm² or less). The larger surface area presented by filter 219 to material flowing through the filtration element helps prevent clogging while avoiding significant increases in the void volume (discussed hereinbelow) of the processing region.

Typically, the total volume (including particles 218) between inlet 265 and filter 219 is about 15 microliters or less (e.g., about 10 microliters or less, about 5 microliters or less, about 2.5 microliters or less, about 2 microliters or less). In an exemplary embodiment, the total volume is about 2.3 microliters. In some embodiments, particles 218 occupy at least about 10 percent (e.g., at least about 15 percent) of the total volume of the filtration element. In some embodiments, particles 218 occupy about 75 percent or less (e.g., about 50 percent or less, about 35 percent or less) of the total volume of processing chamber 220.

In some embodiments, the volume of the filtration element that is free to be occupied by liquid (e.g., the void volume of processing region 220 including interstices between particles 218) is about equal to the total volume minus the volume occupied by the particles. Typically, the void volume of the filtration element is about 10 microliters or less (e.g., about 7.5 microliters or less, about 5 microliters or less, about 2.5 microliters or less, about 2 microliters or less). In some embodiments, the void volume is about 50 nanoliters or more (e.g., about 100 nanoliters or more, about 250 nanoliters or more). In an exemplary embodiment, the total volume of the filtration element is about 2.3 microliters. For example, in an exemplary embodiment, the total volume of the filtration element is about 2.3 microliters, the volume occupied by particles is about 0.3 microliters, and the volume free to be occupied by liquid (void volume) is about 2 microliters.

In some embodiments, a volume of channel 287 between filter 219 and outlet 267 is substantially smaller than the void volume of the filtration element. For example, in some embodiments, the volume of channel 287 between filter 219 and outlet 267 is about 35% or less (e.g., about 25% or less, about 20% or less) of the void volume. In an exemplary embodiment, the volume of channel 287 between filter 219 and outlet 267 is about 500 nanoliters.

Filter 219 typically has pores with a width smaller than the diameter of particles 218. In an exemplary embodiment, filter 219 has pores having an average width of about 8 microns, and particles 218 have an average diameter of about 10 microns.

While the filtration element has been described as having a retention member formed of multiple surface-modified particles, other configurations can be used. For example, in some embodiments, filtration element includes a retention member configured as a porous member (e.g., a filter, a porous membrane, or a gel matrix) having multiple openings (e.g., pores and/or channels) through which polynucleotides pass. Surfaces of the porous member are modified to preferentially retain polynucleotides. Filter membranes available from, for example, Osmonics, are formed of polymers that may be surface-modified and used to retain polynucleotides within processing region 220. In some embodiments, processing region 220 includes a retention member configured as a plurality of surfaces (e.g., walls or baffles) through which a sample passes. The walls or baffles are modified to preferentially retain polynucleotides.

Channels

Channels of microfluidic component 201 typically have at least one sub-millimeter cross-sectional dimension. For example, channels of network 201 may have a width and/or a depth of about 1 mm or less (e.g., about 750 microns or less, about 500 microns, or less, about 250 microns or less) and are at least 1 μm thick, preferably at least 10 μm think, and more preferably at least 100 μm thick. Channels of component 201 typically hold at least about 0.375 microliters of liquid (e.g., at least about 0.750 microliters, at least about 1.25 microliters, at least about 2.5 microliters). In some embodiments, channels hold about 7.5 microliters or less of liquid (e.g., about 5 microliters or less, about 4 microliters or less, about 3 microliters or less).

Valves

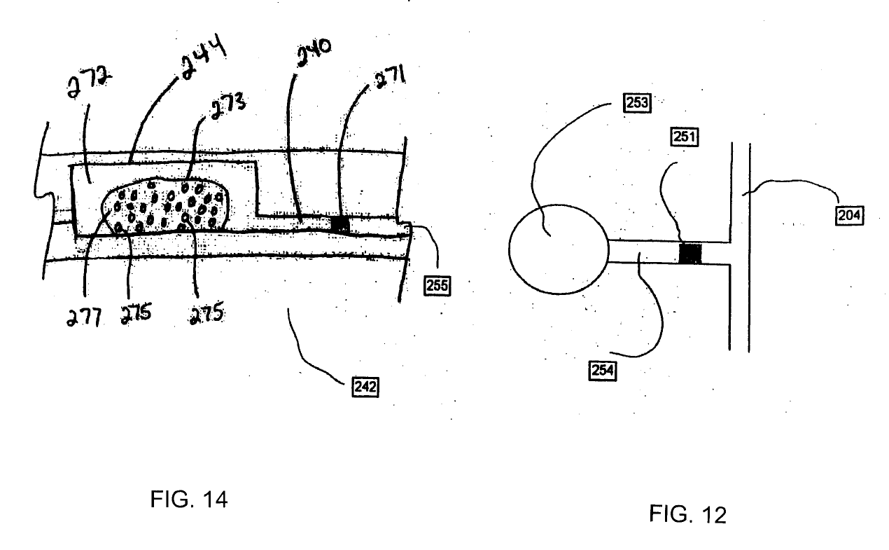

A valve is a component that has a normally open state, allowing material to pass along a channel from a position on one side of the valve (e.g., upstream of the valve) to a position on the other side of the valve (e.g., downstream of the valve). Upon actuation, the valve transitions to a closed state that prevents material from passing along the channel from one side of the valve to the other. For example, valve V1 depicted in FIG. 12 is a single valve that includes a mass 251 of a thermally responsive substance (TRS) that is relatively immobile at a first temperature and more mobile at a second temperature. A chamber 253 is in gaseous communication with mass 251. Upon heating gas (e.g., air) in chamber 253 and heating mass 251 of TRS to the second temperature both utilizing for example a resistive heater in a heater array as shown in FIGS. 7-9, gas pressure within chamber 253 moves mass 251 into channel 204 obstructing material from passing therealong. Other valves of component 201 have the same structure and operate in the same fashion as valve V1.

A mass of TRS can be an essentially solid mass or an agglomeration of smaller particles that cooperate to obstruct the passage. Examples of suitable materials for a TRS include a eutectic alloy (e.g., a solder), wax (e.g., an olefin), polymers, plastics, and combinations thereof. The first and second temperatures are insufficiently high to damage materials, such as polymer layers of cartridge 200. Generally, the second temperature is less than about 90° C., and the first temperature is less than the second temperature (e.g., about 70° C. or less).

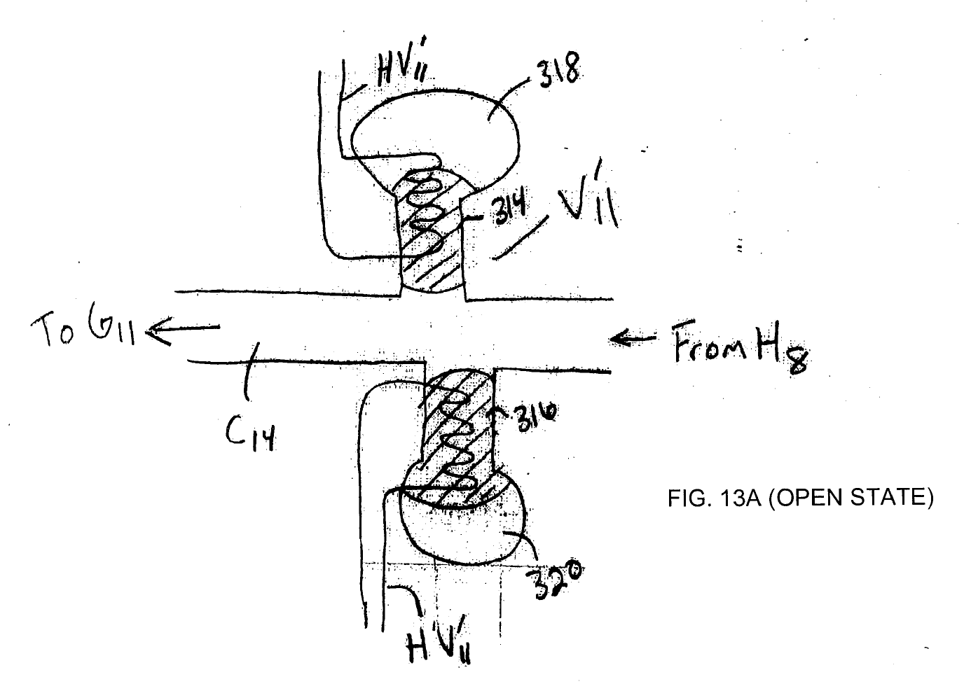

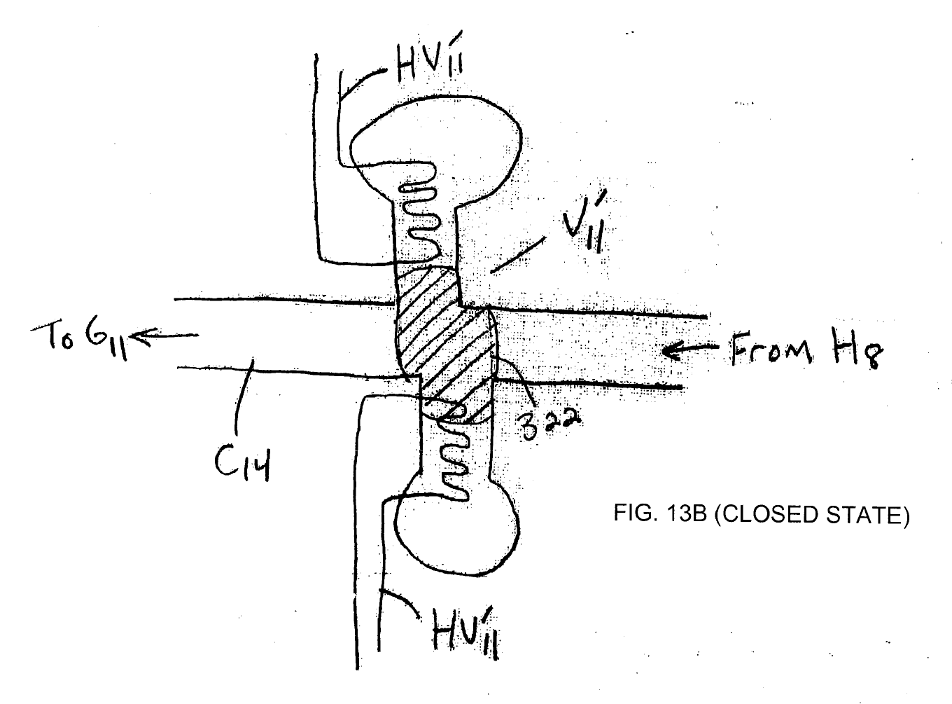

Valves for use with the present invention may be double valves or single valves. As seen in FIGS. 13A and 13B, double valves Vi′ are also components that have a normally open state allowing material to pass along a channel from a position on one side of the valve (e.g., upstream of the valve) to a position on the other side of the valve (e.g., downstream of the valve). Taking double valve V11′ of FIGS. 13A and 13B as an example, double valves Vi′ include first and second masses 314, 316 of a TRS (e.g., a eutectic alloy or wax) spaced apart from one another on either side of a channel. Typically, the TRS masses 314, 316 are offset from one another (e.g., by a distance of about 50% of a width of the TRS masses or less). Material moving through the open valve passes between the first and second TRS masses 314, 316. Each TRS mass 314, 316 is associated with a respective chamber 318, 320, which typically includes a gas (e.g., air).

The TRS masses 314, 316 and chambers 318, 320 of a double valve Vi′ are preferably in thermal contact with a corresponding heat source of a heat source network such as depicted in FIGS. 7-9. Actuating the corresponding heat source causes TRS masses 314, 316 to transition to a more mobile second state (e.g., a partially melted state) and increases the pressure of gas within chambers 318, 320. The gas pressure drives TRS masses 314, 316 across channel C11 and closes valve HV11′ (FIG. 13B). Typically, masses 314, 316 at least partially combine to form a mass 322 that obstructs channel C11.

In order to fit as many as 8 sample lanes or cartridges into a multi-lane cartridge, the double valves may be designed to take up less effective space on the cartridge. This can be achieved by adding bends to the channel containing the TRS.

Gates

A gate is a component that has a normally closed state that does not allow material to pass along a channel from a position on one side of the gate to a position on the other side of the gate. A gate is typically actuated (e.g., opened) to allow pressure created in the chamber of an actuator to enter the microfluidic component. Upon actuation, the gate transitions to an open state in which material is permitted to pass from one side of the gate (e.g., upstream of the gate) to the other side of the gate (e.g., downstream of the gate). An exemplary gate structure is shown in FIG. 12, in connection with an actuator. For example, gate 242 includes a mass 271 of TRS positioned to obstruct passage of material between junction 255 and channel 240. Upon heating mass 271 to the second temperature, the mass changes state (e.g., by melting, by dispersing, by fragmenting, and/or dissolving) to permit passage of material between junction 255 and channel 240.

A gate is typically activated with an actuator in microfluidic devices known in the art. In the present invention, a gate is preferably actuated by pressure from an inlet such as the reagent inlet. An actuator is a component that provides a gas pressure that can move material (e.g., sample material and/or reagent material) between one location of component 201 and another location. For example, referring to FIG. 12, actuator 244 includes a chamber 272 having a mass 273 of thermally expansive material (TEM) therein. When heated, the TEM expands thereby decreasing the free volume within chamber 272 and pressurizing the gas (e.g., air) surrounding mass 273 within chamber 272. In some embodiments, actuator 244 can generate a pressure differential of more than about 3 psi (e.g., at least about 4 psi, at least about 5 psi) between the actuator and junction 255.

The gates of the microfluidic component of the present invention may also be opened from a closed state to an open state by using pressure from an external source. In the present invention, the gates are preferably opened by forcing the various buffers from the reagent inlet by using external pressure provided by the system.

The TEM preferably includes a plurality of sealed liquid reservoirs (e.g., spheres) 275 dispersed within a carrier 277 as shown in FIG. 12. Typically, the liquid is a high vapor pressure liquid (e.g., isobutane and/or isopentane) sealed within a casing (e.g., a polymeric casing formed of monomers such as vinylidene chloride, acrylonitrile and methylmethacrylate). Carrier 277 has properties (e.g., flexibility and/or an ability to soften (e.g., melt) at higher temperatures) that permit expansion of the reservoirs 275 without allowing the reservoirs to pass along channel 240. In some embodiments, carrier 277 is a wax (e.g., an olefin) or a polymer with a suitable glass transition temperature. Typically, the reservoirs make up at least about 25 weight percent (e.g., at least about 35 weight percent, at least about 50 weight percent) of the TEM. In some embodiments, the reservoirs make up about 75 weight percent or less (e.g., about 65 weight percent or less, about 50 weight percent or less) of the TEM. Suitable sealed liquid reservoirs can be obtained from Expancel (Akzo Nobel).

When the TEM is heated (e.g., to a temperature of at least about 50° C. (e.g., to at least about 75° C., at least about 90° C.)), the liquid vaporizes and increases the volume of each sealed reservoir and of mass 273. Carrier 277 softens, allowing mass 273 to expand. Typically, the TEM is heated to a temperature of less than about 150° C. (e.g., about 125° C. or less, about 110° C. or less, about 100° C. or less) during actuation. In some embodiments, the volume of the TEM expands by at least about 5 times (e.g., at least about 10 times, at least about 20 times, at least about 30 times).

Gates for use with the present invention may be simple gates, or mixing gates. Mixing gates are components that allow two volumes of liquid to be combined (e.g., mixed).

Vents

A vent is a structure that permits gas (e.g., air), such as gas displaced by the movement of liquids within component 201, to exit a channel while simultaneously limiting (e.g., preventing) liquid from exiting the channel. Vents thus allow component 201 to be vented so that pressure buildup does not inhibit desired movement of the liquids.

Typically, a vent is a hydrophobic vent and includes a layer of porous hydrophobic material (e.g., a porous filter such as a porous hydrophobic membrane, available from Osmonics) that defines a wall of the channel. As discussed hereinbelow, hydrophobic vents can be used to position a microdroplet of sample at a desired location within component 201.

Hydrophobic vents typically have a length of at least about 2.5 mm (e.g., at least about 5 mm, at least about 7.5 mm) along a channel. The length of a hydrophobic vent is typically at least about 5 times (e.g., at least about 10 times, at least about 20 times) larger than a depth of the channel within the hydrophobic vent. For example, in some embodiments, the channel depth within the hydrophobic vent is about 300 microns or less (e.g., about 250 microns or less, about 200 microns or less, about 150 microns or less).

The depth of the channel within the hydrophobic vent is typically about 75% or less (e.g., about 65% or less, about 60% or less) of the depth of the channel upstream and downstream of the hydrophobic vent. For example, in some embodiments the channel depth within the hydrophobic vent is about 150 microns and the channel depth upstream and downstream of the hydrophobic vent is about 250 microns.

A width of the channel within the hydrophobic vent is typically at least about 25% wider (e.g., at least about 50% wider) than a width of the channel upstream from the vent and downstream from the vent. For example, in an exemplary embodiment, the width of the channel within the hydrophobic vent is about 400 microns and the width of the channel upstream and downstream from the vent is about 250 microns.

Waste Chambers

Waste chambers are elements that can receive waste (e.g., overflow) liquid resulting from the manipulation (e.g., movement and/or mixing) of liquids within microfluidic component. Typically, each waste chamber has an associated air vent that allows gas displaced by liquid entering the chamber to be vented. An exemplary waste chamber is shown at 269 in FIG. 4.

System

Elements of component 201 are typically thermally actuated. Accordingly, in use, cartridge 200 is typically in communication with a heating element, such as an array of heat sources (e.g., resistive heat sources as exemplified in FIGS. 7-9), configured to operate the elements (e.g., valves, gates, actuators, and processing region) of microfluidic component 201. By ‘in communication’, is included to mean thermally associated, for example in thermal contact with a heat source. In preferred embodiments, cartridge 200 is insertable into, and removable from, a cartridge receiving element in a system such as shown in FIG. 1. The heating element is in communication with the cartridge receiving element and is configured to heat one or more regions of the cartridge.

In some embodiments, the heat sources are operated by a computer operating system, which operates the device during use by communicating instructions to various control circuitry that is in communication with the heating element. The operating system includes a processor (e.g., a computer) configured to actuate the heat sources at specific times, according to a desired protocol. Processors configured to operate microfluidic devices are described in U.S. application Ser. No. 09/819,105, filed Mar. 28, 2001, which is incorporated herein by reference. In other embodiments, the heat sources are integral with the system itself.

Preferably, heat sources in the array of heat sources have locations that correspond to elements, such as actuators, gates, and valves, of microfluidic component 201.

Lyophilized Particles

Lyophilized reagent pellets 260 of bulk lysis chamber 264 include one or more compounds (e.g., reagents) configured to release polynucleotides from cells (e.g., by lysing the cells). For example, pellets can include one or more enzymes configured to reduce (e.g., denature) proteins (e.g., proteinases, proteases (e.g., pronase), trypsin, proteinase K, phage lytic enzymes (e.g., PlyGBS)), lysozymes (e.g., a modified lysozyme such as ReadyLyse), cell specific enzymes (e.g., mutanolysin for lysing group B streptococci)).

The pellets generally have a room temperature (e.g., about 20° C.) shelf-life of at least about 6 months (e.g., at least about 12 months). Liquid sample entering the bulk lysis chamber dissolves (e.g., reconstitutes) the lyophilized compounds.

Typically, pellets 264 have an average volume of about 35 microliters or less (e.g., about 27.5 microliters or less, about 25 microliters or less, about 20 microliters or less). In some embodiments, the particles have an average diameter of about 8 mm or less (e.g., about 5 mm or less, about 4 mm or less) In an exemplary embodiment the lyophilized pellets have an average volume of about 20 microliters and an average diameter of about 3.5 mm.

In some embodiments, pellets alternatively or additionally include components for retaining polynucleotides as compared to inhibitors. For example, pellets 260 can include multiple pellets surface modified with ligands, as discussed hereinabove. Pellets 260 can include enzymes that reduce polynucleotides that might compete with a polynucleotide to be determined for binding sites on the surface modified particles. For example, to reduce RNA that might compete with DNA to be determined, pellets 260 may include an enzyme such as an RNAase (e.g., RNAseA ISC BioExpress (Amresco)).

In an exemplary embodiment, pellets 260 cells include a cryoprotecant. Cryoprotectants generally help increase the stability of the lypophilized particles and help prevent damage to other compounds of the particles (e.g., by preventing denaturation of enzymes during preparation and/or storage of the particles). In some embodiments, the cryoprotectant includes one or more sugars (e.g., one or more dissacharides (e.g., trehalose, melizitose, raffinose)) and/or one or more poly-alcohols (e.g., mannitol, sorbitol).

Lyophilized particles can be prepared as desired. Typically, the particles are prepared using a cryoprotectant and chilled hydrophobic surface. Typically, compounds of the lyophilized particles are combined with a solvent (e.g., water) to make a solution, which is then placed (e.g., in discrete aliquots (e.g., drops) such as by pipette) onto a chilled hydrophobic surface (e.g., a diamond film or a polytetrafluorethylene surface). In general, the temperature of the surface is reduced to near the temperature of liquid nitrogen (e.g., about −150° F. or less, about −200° F. or less, about −275° F. or less). The solution freezes as discrete particles. The frozen particles are subjected to a vacuum while still frozen for a pressure and time sufficient to remove the solvent (e.g., by sublimation) from the pellets.

For example, a solution for preparing particles can be prepared by combining a cryoprotectant (e.g., 6 grams of trehalose), a plurality of particles modified with ligands (e.g., about 2 milliliters of a suspension of carboxylate modified particles with poly-D-lysine ligands), a protease (e.g., 400 milligrams of pronase), an RNAase (e.g., 30 milligrams of RNAseA (activity of 120 U per milligram), an enzyme that digests peptidoglycan (e.g., ReadyLyse (e.g., 160 microliters of a 30000 U per microliter solution of ReadyLyse)), a cell specific enzyme (e.g., mutanolysin (e.g., 200 microliters of a 50 U per microliter solution of mutanolysin), and a solvent (e.g., water) to make about 20 milliters. About 1,000 aliquots of about 20 microliters each of this solution are frozen and desolvated as described above to make 1,000 pellets. When reconstituted, the pellets are typically used to make a total of about 200 milliliters of solution.

In general, the concentrations of the compounds in the solution from which the particles are made is higher than when reconstituted in the microfluidic device. Typically, the ratio of the solution concentration to the reconstituted concentration is at least about 3 (e.g., at least about 4.5). In some embodiments, the ratio is about 6.

Operation

In an exemplary embodiment, cartridge 200 may be operated as shown in FIGS. 4 and 15-27, and as described as follows. It is to be understood that these figures depict an exemplary embodiment and that other embodiments are within the scope of the present invention, for example the exemplary operation described in FIGS. 6-17 of U.S. provisional application Ser. No. 60/726,066, filed Oct. 11, 2005, and incorporated herein by reference in its entirety.

Prior to sample processing, valves of component 201 are configured in the open state, and gates of component 201 are configured in the closed state.

Approximately 1.5 milliliters of clinical sample 600, in fluid form, is input into bulk lysis chamber 264 through sample inlet 202. For example, sample can be introduced with a syringe having a fitting complementary to a luer on sample inlet 202. In other embodiments, the amount of sample introduced is about 500 microliters or less (e.g., about 250 microliters or less, about 100 microliters or less, about 50 microliters or less, about 25 microliters or less, about 10 microliters or less). In some embodiments, the amount of sample is about 2 milliliters or less (e.g., 1.5 milliliters or less).

An excess amount of air (about 1-3 ml and typically 2.5 ml) of air is also injected into the sealed bulk lysis chamber, through sample inlet 202 preferably at the same time that the sample is injected. The air above the fluid sample is under compression during this stage until the pressure is released later on.

The liquid sample dissolves the bulk lysis reagent pellets and capture reagent pellets in the lysis chamber 264, if present. Reconstituted lysing reagents (e.g., ReadyLyse, mutanolysin) begin to lyse cells of the sample releasing polynucleotides. Other reagents (e.g., protease enzymes such as pronase) begin to reduce or denature inhibitors (e.g., proteins) within the sample. Polynucleotides from the sample begin to associate with (e.g., bind to) ligands of particles released from the pellets.

The cartridge is placed in the cartridge receiving element of a system such as system 10, FIG. 1, either after the sample is introduced of before. The user instructs the system to proceed with sample preparation, say by delivering appropriate instructions through a user interface 32. In preferred embodiments, the system begins sample preparation automatically after the cartridge receiving element has accepted a cartridge and has communicated its acceptance to a controller.

The sample in the bulk lysis chamber is heated up to a temperature sufficient to initiate chemical lysis of the cells. Lysing of cells may occur by application of heat alone, or by a combination of heat and lysis reagents, as described herein. The chamber is typically at a temperature of about 50° C. or less (e.g., 30° C. or less) during introduction of the sample. Typically, the sample within chamber 264 is heated to a temperature in the range 60-80° C. (e.g., to at least about 65° C., to at least about 70° C.) for a short period of time, preferably 5-10 minutes, (e.g., for about 15 minutes or less, about 10 minutes or less, about 7 minutes or less) while lysing occurs.

In some embodiments, a heat lamp in close proximity to the bulk lysis chamber, heats the sample. In other embodiments, optical energy is used at least in part to heat contents of lysing chamber 264. For example, the operating system used to operate device 300 can include a light source (e.g., a lamp primarily emitting light in the infrared) disposed in thermal and optical contact with chamber 264. An especially preferred manner of heating is by contact heating, such as by direct contact of a heating element with upper surface 266 of the lysis chamber, as accomplished by exemplary system 10 of FIG. 1. Chamber 264 preferably includes a temperature sensor used to monitor the temperature of the sample within chamber 264. The heat output of the heat source is increased or decreased based on the temperature determined with sensor.

The bulk lysis reagents contain a cocktail of reagents that chew up the cell walls of the target cells, chew up PCR inhibiting proteins, lipids, etc., and also have DNA (or RNA) affinity beads (˜10 micron in median diameter) that capture DNA (or RNA) present in the sample. This process typically takes between about 1 and about 5 minutes.

Polynucleotides of the sample contacting the affinity beads are preferentially retained as compared to liquid of the sample and certain other sample components (e.g., inhibitors). Typically, the affinity beads retain at least about 50% of polynucleotides (at least about 75%, at least about 85%, at least about 90%) of the polynucleotides present in the sample that entered processing region 220.

After completion of lysis and capture of DNA onto reagent beads, the lysed sample flows through hole H1 and into the microfluidic component, as shown in FIG. 15. Hole H1 is always open to permit sample to flow through but passage of sample is effectively controlled by gate G1 and thus sample does not exit through H1 until G1 is opened. The sample flows past valve V1, junction 259 and along channel 239 towards capture filter C. Motion towards gate G5 is impeded.

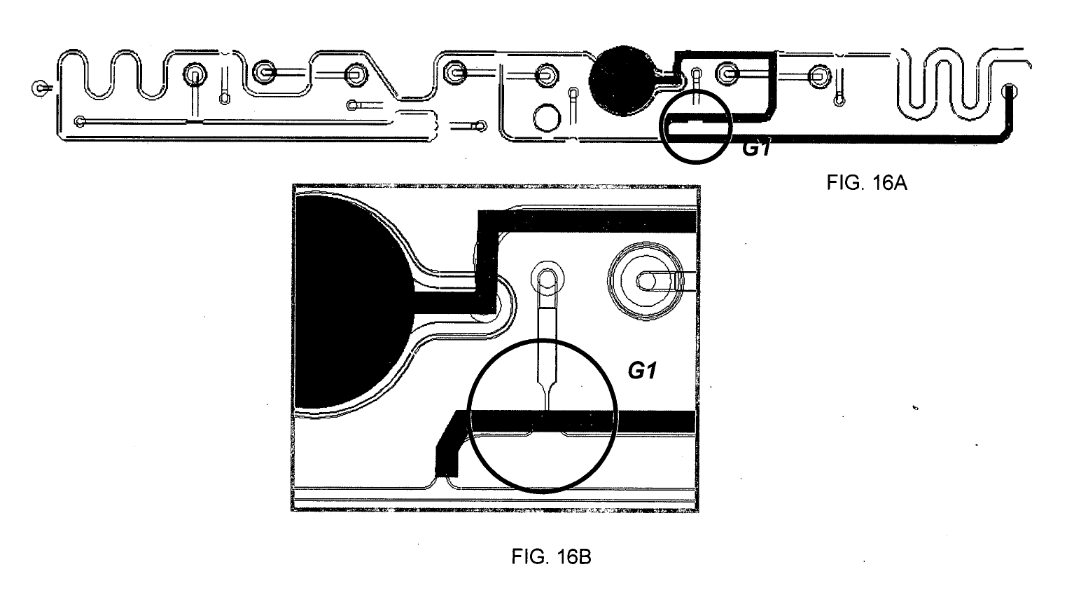

Gate G1 is opened, for example by heating, and continual expansion of air from chamber 264 forces the sample to flow along channel 239 to capture filter C. Pressure within chamber 264 drives the lysed sample material (containing lysate, polynucleotides bound to particles, and other sample components) along the pathway. During this flow, as depicted in FIGS. 16A and 16B, the DNA capture beads get trapped at the inline filter (C.). Preferably filter C. is a 8 micron filter. Valve V2 has remained open during this process.

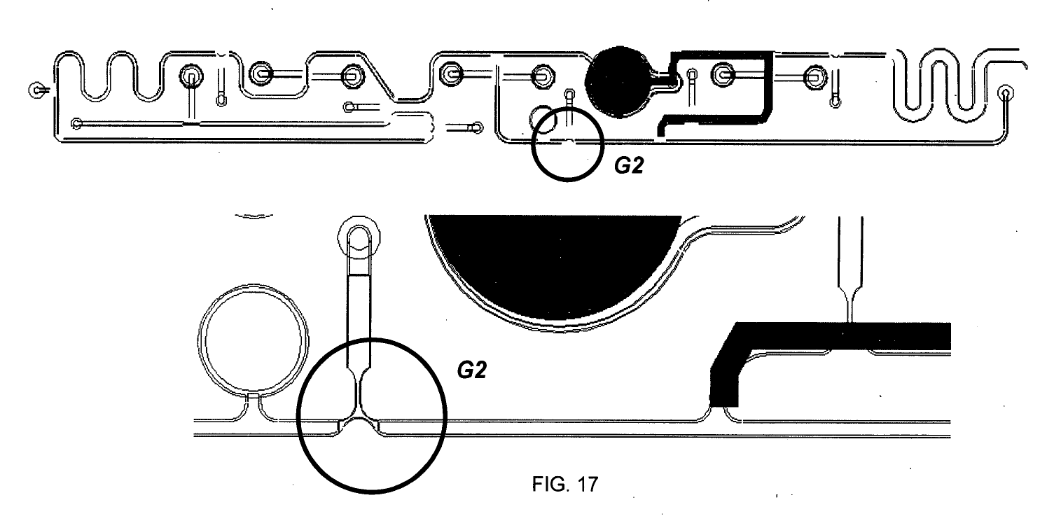

Next, after a period of time (e.g., between about 2 and about 5 minutes), as depicted in FIG. 17, the excess pressure in the bulk lysis chamber is vented to atmosphere through hole H4 to the waste chamber by opening gate G1.

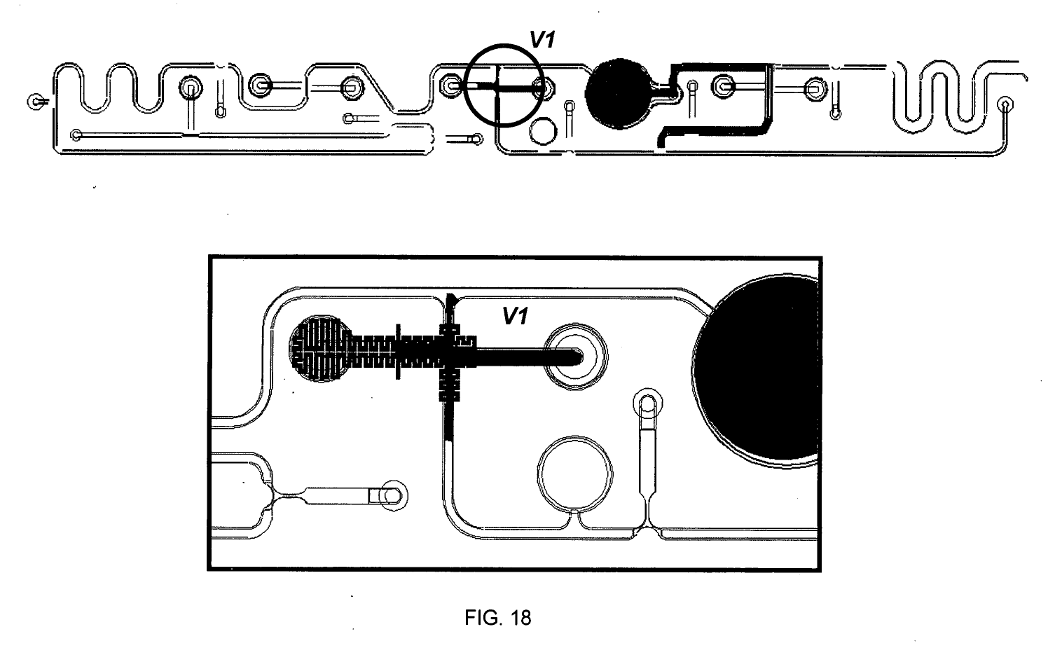

Valve V1 is now closed, as shown in FIG. 18, to prevent any liquid leaking back into the bulk lysis chamber during further liquid processing, and thereby sealing off the lysis chamber.

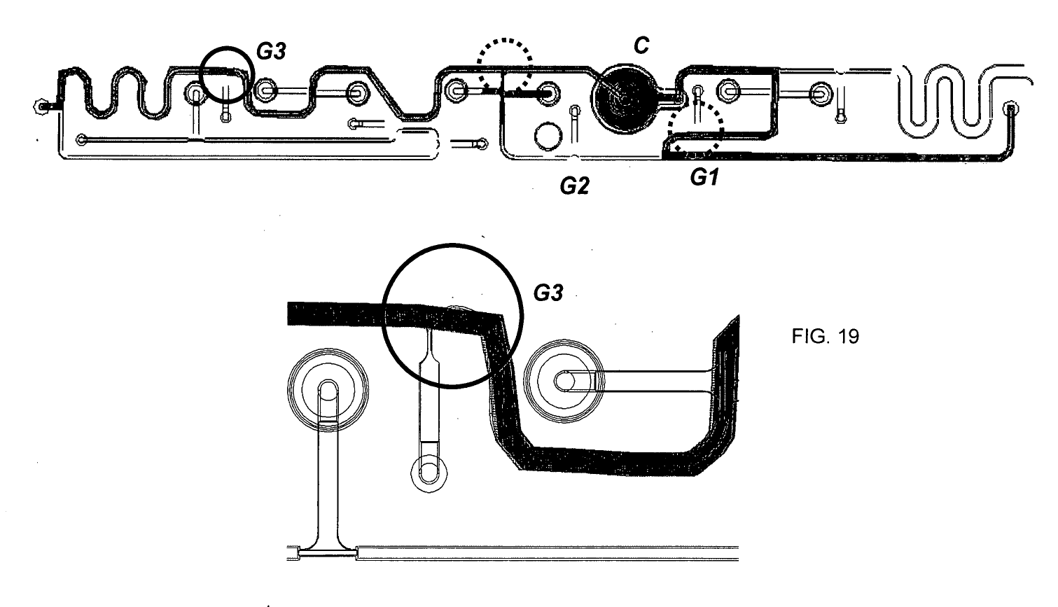

In a next step, wash reagent is input, preferably automatically by a system such as system 10, through the pierceable inlet 280 and via hole H1, forced through channels 208, 211, 213, 239, 210, 236, and 234, along the shaded flow path in FIG. 19, to wash the filter, C. Gates G1 and G3 are opened to open this flow path, whereas G2 and V1 remain closed. Typically, the wash liquid is a solution having one or more additional components (e.g., a buffer, chelator, surfactant, a detergent, a base, an acid, or a combination thereof). A typical volume of wash buffer used in this step is 50 μl. Exemplary solutions include those, for example, made from a solution of 10-50 mM Tris at pH 8.0, 0.5-2 mM EDTA, and 0.5%-2% SDS, a solution of 10-50 mM Tris at pH 8.0, 0.5 to 2 mM EDTA, and 0.5%-2% Triton X-100.

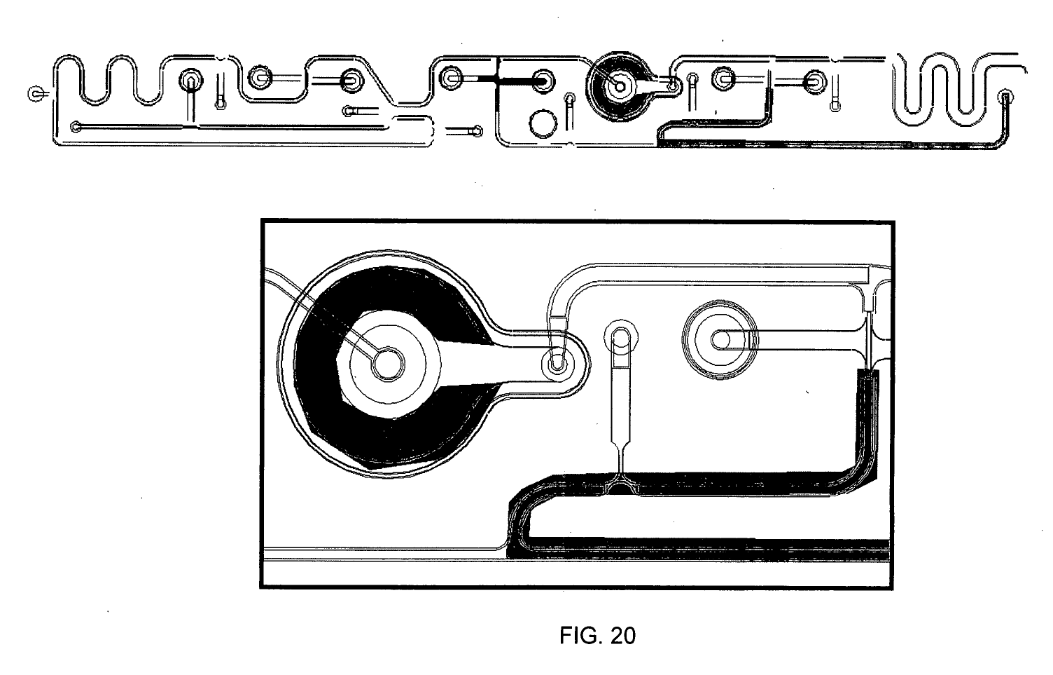

Thereafter, FIG. 20, the bead column is purged with air by introducing air, for example between 10 and 100 μl of air, through the reagent inlet. The result is a purging of wash buffer through hole H4 into the waste chamber.

Next, in FIG. 21, release buffer is input from the reagent inlet 280 to replace the wash solution, and the end terminus of the release buffer liquid volume passes through column C. An exemplary release liquid is a hydroxide solution (e.g., a NaOH solution) having a concentration of, for example, between about 2 mM hydroxide (e.g., about 2 mM NaOH) and about 500 mM hydroxide (e.g., about 500 mM NaOH). In some embodiments, liquid in reservoir 281 is an hydroxide solution having a concentration of about 25 mM or less (e.g., an hydroxide concentration of about 15 mM). A typical volume of release buffer is 50 μl.

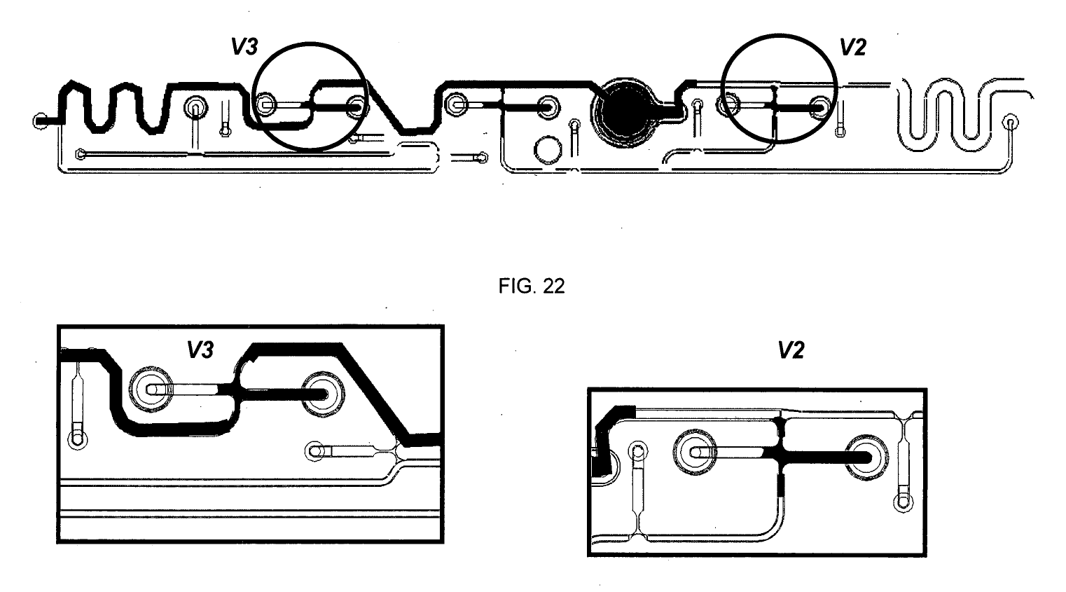

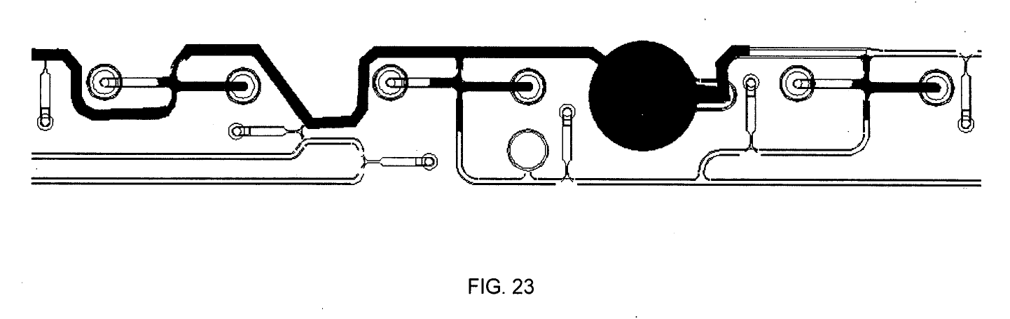

Valves V2 and V3 are now closed, to seal off the column C, as shown in FIG. 22. The bead column C is heated to 70-90° C. for 3-4 minutes to release the DNA from the affinity beads in the presence of release buffer, FIG. 23.

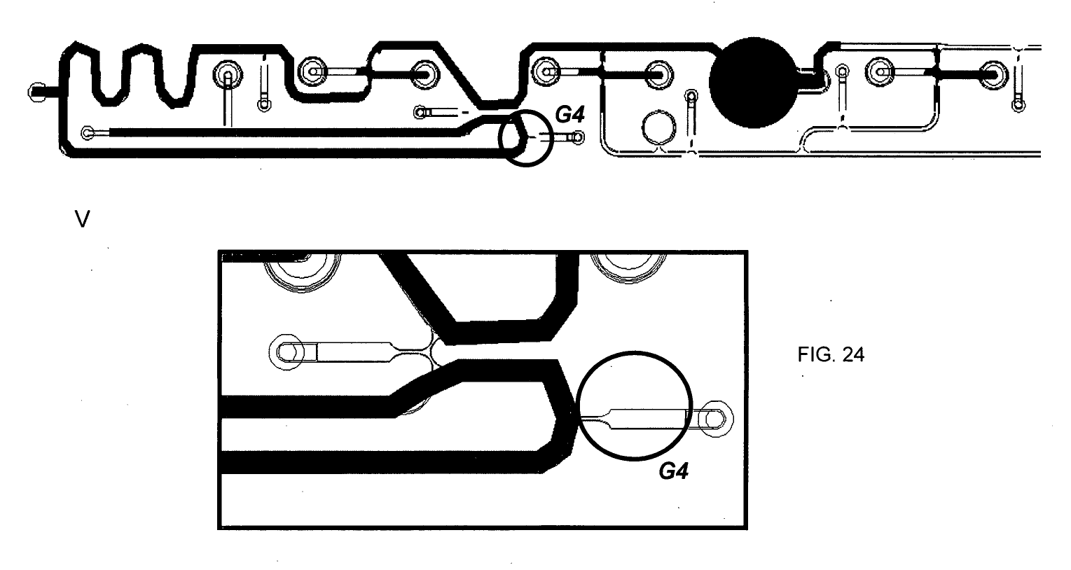

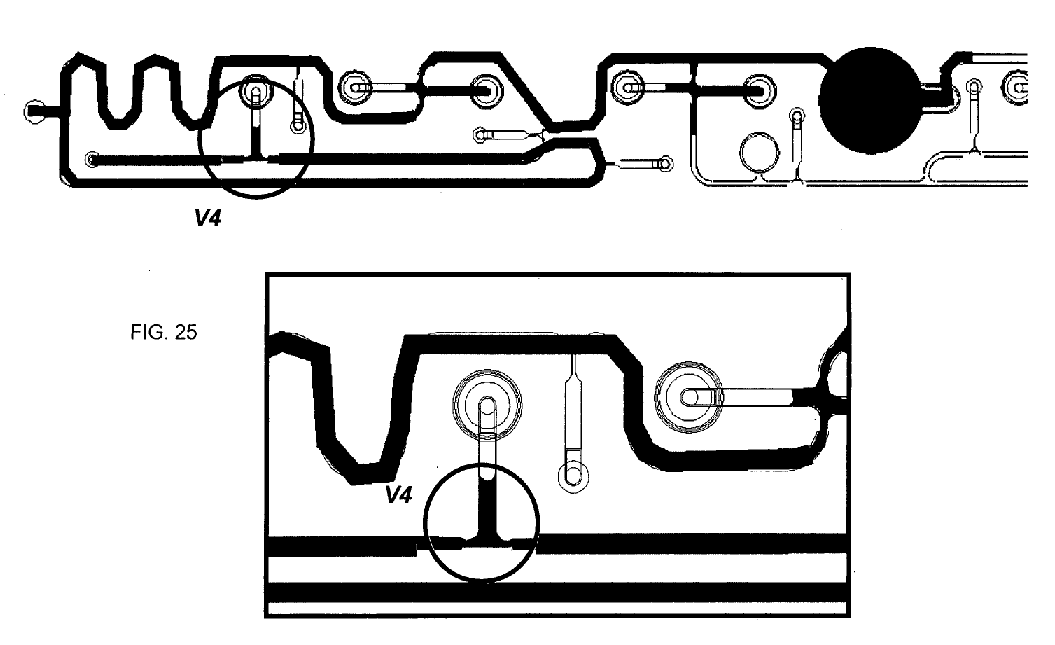

Neutralization buffer (about 5 μl) is next input through the reagent inlet, and sent to the vent V by opening gate G4, as shown in FIG. 24. Valve V4 is now closed, FIG. 25.

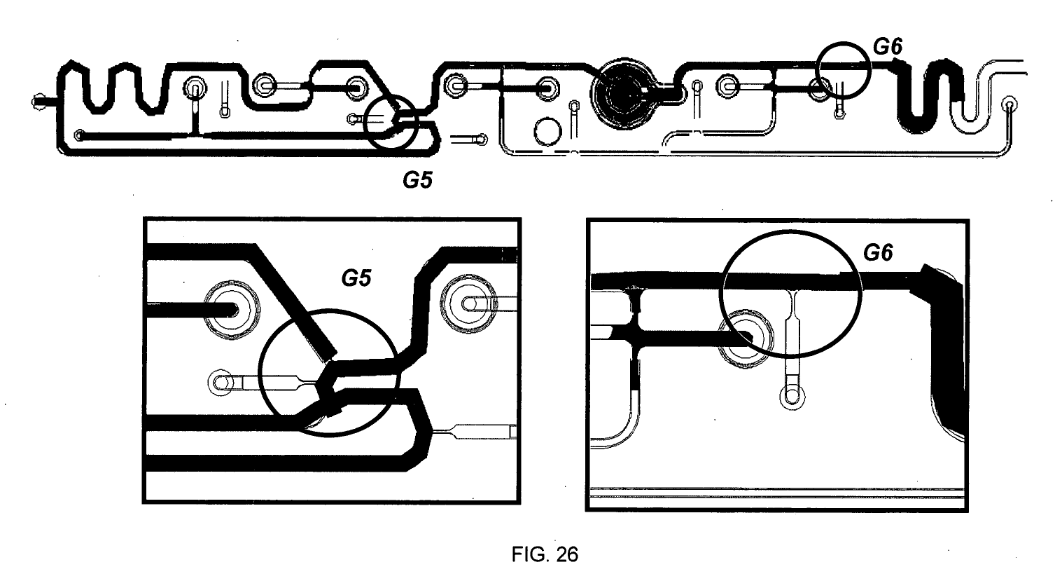

A further 0-45 μl of neutralization buffer is pumped into the microfluidic component through inlet 280, and mixed with released DNA by opening gates G4, G5, and G6, as shown in FIG. 26.

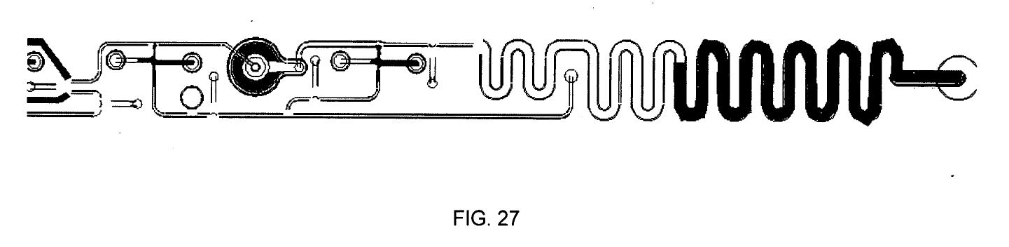

Upon input from the user, air is again pumped through the reagent inlet, and gates G5 and G6 are opened to combine neutralization buffer with the released DNA. This step is not generally automated because it is preferred to start the reaction in a controlled manner. The mixture is pumped through a specified channel volume, using for example pressurized air transmitted through the reagent inlet, to intermix and neutralize the DNA, before ejecting the mixed sample into a PCR tube, as shown in FIG. 27.

The neutralized DNA (or RNA) is forced into the PCR tube at the end of the sample processing unit. The liquid in which the polynucleotides are released into a PCR tube typically includes at least about 50% (e.g., at least about 75%, at least about 85%, or at least about 90%) of the polynucleotides present in the sample that was introduced into the bulk lysis chamber. The concentration of polynucleotides present in the release liquid may be higher than in the original sample because the volume of release liquid is typically less than the volume of the original liquid sample. For example the concentration of polynucleotides in the release liquid may be at least about 10 times greater (e.g., at least about 25 times greater, at least about 100 times greater) than the concentration of polynucleotides in the sample introduced to device 200. The concentration of inhibitors present in the liquid into which the polynucleotides are released is generally less than concentration of inhibitors in the original fluidic sample by an amount sufficient to increase the amplification efficiency for the polynucleotides.

The time interval between introducing the polynucleotide containing sample to the bulk lysis chamber, and releasing the polynucleotides into the PCR tube is typically about 15 minutes or less (e.g., about 10 minutes or less, about 5 minutes or less). The PCR tube containing PCR-ready DNA is ready for further processing in a bench scale PCR detection machine, and can thus be removed.

A number of embodiments have been described. Nevertheless, it will be understood that various modifications may be made without departing from the spirit and scope of the disclosure. Accordingly, other embodiments are within the scope of the following claims.

What is claimed is:

1. A microfluidic system for converting a sample containing one or more polynucleotides into a form suitable for analyzing the one or more polynucleotides, the system comprising:
a cartridge receiving element in communication with an insertable and removable cartridge;
a heating element in communication with the cartridge receiving element, configured to heat one or more regions of the cartridge; and
control circuitry in communication with the heating element;
wherein the insertable cartridge comprises:
at least one microfluidic component that, in conjunction with the heating element and the control circuitry, is configured to accept the sample and one or more reagents, and to react the sample and the reagents, in order to produce a prepared sample suitable for analysis of the one or more polynucleotides.

2. The system of claim 1, wherein the insertable cartridge further comprises:
a sample inlet for receiving the sample;
a reagent inlet for accepting one or more reagents; and
an outlet for directing prepared sample into a PCR tube.

3. The system of claim 2, wherein the microfluidic component comprises:
one or more channels configured to transmit volumes of fluid in the range 0.1-50 μl, wherein the one or more channels ensure passage of sample, reagents, and fluid between the sample inlet, the reagent inlet, and the outlet.

4. The system of claim 1, wherein the microfluidic component comprises one or more microfluidic elements selected from the group consisting of:
at least one valve;
at least one gate;
at least one filter; and
at least one waste chamber.

5. The system of claim 4, wherein one or more of the at least one valves is situated in one of the regions of the cartridge that is heated by the heating element, and comprises a material that melts when the heating element applies heat thereto.

6. The system of claim 1, wherein the analyzing is performed by a machine configured to carry out a method selected from the group consisting of: PCR, TMA, SDA, and NASBA.

7. The system of claim 1 wherein the sample is between about 0.5 mL and 2.0 mL in volume.

8. The system of claim 2 further comprising a heating element for heating the sample in the sample inlet.

9. The system of claim 1, further comprising a display that communicates to a user of the system one or more of:
current status of the system;
progress of sample preparation; and
a warning message in case of malfunction of either system or cartridge.

10. The system of claim 1, further comprising an interface for connecting the system to a computer or a network of computers.

11. The system of claim 1, further comprising a computer-readable memory which stores instructions for operating the control circuitry.

12. The system of claim 11 further comprising a processing unit for executing the instructions.

13. The system of claim 1 further comprising an input device for accepting information from a user.

14. The system of claim 1, wherein the cartridge is configured to accept two or more separate samples.

15. The system of claim 1, configured to accept two or more cartridges.

16. The system of claim 15, configure to accept three cartridges.

17. A microfluidic cartridge for converting a sample containing one or more polynucleotides into a form suitable for analyzing the one or more polynucleotides, the cartridge comprising:
a sample inlet for receiving the sample;
a reagent inlet for accepting one or more reagents;
an outlet for directing prepared sample into a PCR tube; and
a microfluidic component having one or more channels configured to transmit volumes of fluid in the range 0.1-50 μl;
wherein the one or more channels ensure passage of sample, reagents, and fluid between the sample inlet, the reagent inlet, and the outlet; and
wherein the microfluidic cartridge, in conjunction with an external source of heat, is configured to react the sample and the reagents, in order to produce a prepared sample suitable for analyzing the one or more polynucleotides.

18. The microfluidic cartridge of claim 17, wherein the PCR tube is removable.

19. A multi-sample cartridge for converting a number of samples, including at least a first sample and a second sample, wherein said first sample and said second sample each contain one or more polynucleotides, into respective forms suitable for analyzing the one or more polynucleotides, the multi-sample cartridge comprising:
at least a first microfluidic cartridge and a second microfluidic cartridge,
separably affixed to one another, wherein each of said first microfluidic cartridge and said second microfluidic cartridge is according to claim 15, and wherein the first microfluidic cartridge accepts the first sample, and wherein the second microfluidic cartridge accepts the second sample.

20. The multi-sample cartridge of claim 19, wherein said number is eight.

21. The multi-sample cartridge of claim 19 having a size substantially the same as that of a 96-well plate.

22. The multi-sample cartridge of claim 19, further comprising a first PCR tube attached to the first microfluidic component, and a second PCR tube attached to the second microfluidic component.

23. The multi-sample cartridge of claim 22, wherein the first sample is converted into a first prepared sample, delivered to the first PCR tube, and the second sample is converted into a second prepared sample, delivered to the second PCR tube.

24. The multi-sample cartridge of claim 22, wherein the first PCR tube and the second PCR tube are at a distance of 9 mm apart from one another, wherein the distance is measured between a centroid of the first PCR tube and a centroid of the second PCR tube.

25. The multi-sample cartridge of claim 22, wherein the first PCR tube and the second PCR tube are attached to a removable strip.

26. A method of converting a sample comprising a number of cells that have one or more polynucleotides into a form suitable for analyzing the one or more polynucleotides, the method comprising:
introducing from about 0.1-2.0 mL of the sample and an excess quantity of air into a bulk lysis chamber;
applying heat to the sample in the bulk lysis chamber, to raise the sample to a first temperature, thereby lysing cells in the sample and producing a lysate containing the one or more polynucleotides;
capturing one or more polynucleotides in the lysate on an affinity matrix;
causing the beads to leave the bulk lysis chamber and be trapped on a filter;
washing the beads with a wash reagent;
displacing the wash reagent with a release buffer;
heating the beads to a second temperature, thereby releasing the one or more polynucleotides; and
causing the one or more polynucleotides to be transferred to a PCR tube.

27. The method of claim 26, wherein prior to applying heat to the sample, the sample is dissolved in one or more lysis reagents in the bulk lysis chamber.

28. The method of claim 26 wherein the affinity matrix comprises one or more beads.

29. The method of claim 26 further comprising, after heating the beads to the second temperature:
combining a neutralization buffer with the one or more polynucleotides to produce one or more neutralized polynucleotides; and
wherein the one or more neutralized polynucleotides are transferred to a PCR tube.

30. The method of claim 26, wherein the first temperature is between about 55 and 65° C.

31. The method of claim 26, wherein the second temperature is about 70-95° C.

32. The method of claim 26 wherein the beads comprise poly-lysine or polyethyleneimine.

33. The method of claim 26 wherein the beads are microspheres.

34. The method of claim 26 wherein the sample is kept at the first temperature for up to about 7 minutes.

35. The method of claim 27, wherein the lysis reagents are in the form of one or more lyophilized pellets.

36. The method of claim 28 wherein the one or more beads are in the form of one or more lyophilized pellets.

37. The method of claim 26 wherein the bulk lysis chamber and the PCR tube are part of a microfluidic component.

38. A method of analyzing a sample comprising a number of cells that have one or more polynucleotides, the method comprising:
converting the sample into a form suitable for analyzing the one or more polynucleotides, using the method of claim 24; and
analyzing the sample, using a method selected from the group consisting of: PCR, TMA, SDA, and NASBA.
